(19) United States
(12) Patent Application Publication
(10) Pub. No.: US 2009/0222952 A1
(43) Pub. Date: Sep. 3, 2009

(54) Plants Having Improved Growth Characteristics And A Method For Making The Same

(75) Inventors: Jan de Meutter, Mariakerke (BE); Vladimir Mironov, Gent (BE); Christophe Reuzeau, Tocane (FR); Dirk Inze, Moorsel-Aalst (BE); Kobe Vlieghe, Aalter (BE); Lieven de Veylder, Drongen (BE)

Correspondence Address: CONNOLLY BOVE LODGE & HUTZ, LLP, P O BOX 2207, WILMINGTON, DE, 19899, US

(73) Assignees: CropDesign N.V., Zwijnaarde (BE); VIB vzw, Zwijnaarde (BE); Universiteit Gent, Gent (BE)

(21) Appl. No.: 12/092,844

(22) PCT Filed: Nov. 8, 2006
(86) PCT No.: PCT/EP2006/068248

§ 371 Date: Sep. 15, 2008

Related U.S. Application Data
(60) Provisional application No. 60/734,262, filed on Nov. 8, 2005.
(60) Provisional application No. 60/734,281, filed on Nov. 8, 2005.

(30) Foreign Application Priority Data
Nov. 8, 2005 (EP) 05110458.6
Nov. 8, 2005 (EP) 05110470.1

(51) Int. Cl. A01H 5/00; C12N 15/82; C12N 15/63; C07K 14/415; C12Q 1/68

(52) U.S. Cl. 800/287; 800/278; 800/298; 800/320.2; 800/320.1; 800/320.3; 800/320; 435/320.1; 530/370; 435/6

(57) ABSTRACT

The present invention concerns a method for increasing plant yield and/or increased stress resistance by decreasing the level of activity of a DEL1 polypeptide. One such method comprises introducing into a plant a nucleic acid comprising a variant DEL1 nucleic acid. Another method comprises downregulating expression of a DEL1 gene. The invention also relates to transgenic plants having introduced therein a variant DEL1 nucleic acid thereof, which plants have increased yield and/or increased stress resistance relative to control plants. The present invention also concerns a novel DEL1 protein and its encoding sequence, and constructs useful in the methods of the invention.

The present invention relates generally to the field of molecular biology and concerns a method for improving plant growth characteristics relative to control plants. More specifically, the present invention concerns a method for increasing plant yield and/or increasing stress resistance comprising decreasing the level of activity of a DEL1 polypeptide. The present invention also concerns plants having decreased activity of a DEL1 polypeptide, which plants have increased yield and/or increased stress resistance relative to control plants. Preferably the decreased activity of a DEL1 polypeptide is obtained by modulating expression of a nucleic acid encoding a fragment of a DEL1 polypeptide, or by downregulating expression of a DEL1 gene or a variant thereof. The invention also provides a novel DEL1 polypeptide, nucleic acids encoding such a polypeptide, and constructs useful in the methods of the invention.

The ever-increasing world population and the dwindling supply of arable land available for agriculture fuels research towards improving the efficiency of agriculture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic or horticultural traits. A trait of particular economic interest is yield. Yield is normally defined as the measurable produce of economic value from a crop. This may be defined in terms of quantity and/or quality. Yield is directly dependent on several factors, for example, the number and size of the organs, plant architecture (for example, the number of branches), seed production and more. Root development, nutrient uptake and stress tolerance may also be important factors in determining yield. Optimizing one of the abovementioned factors may therefore contribute to increasing crop yield.

Seed yield is a particularly important trait, since the seeds of many plants are important for human and animal nutrition. Crops such as, corn, rice, wheat, canola and soybean account for over half the total human caloric intake, whether through direct consumption of the seeds themselves or through consumption of meat products raised on processed seeds. They are also a source of sugars, oils and many kinds of metabolites used in industrial processes. Seeds contain an embryo (the source of new shoots and roots) and an endosperm (the source of nutrients for embryo growth during germination and during early growth of seedlings). The development of a seed involves many genes, and requires the transfer of metabolites from the roots, leaves and stems into the growing seed. The endosperm, in particular, assimilates the metabolic precursors of carbohydrates, oils and proteins and synthesizes them into storage macromolecules to fill out the grain.

Abiotic stress conditions, such as shortage or excess of solar energy, water and nutrients, salinity, high and low temperature and pollution (e.g., heavy metals), can have a major impact on plant growth and can significantly reduce the yield of, e.g., crop cultivars. Although light is essential for plants since it provides energy for metabolism, UV light is damaging to living organisms. In particular, UV-B radiation (280-320 nm) affects protein and nucleic acids stability resulting in reduced photosynthesis, biomass reduction, decreased protein synthesis and impaired chloroplast function, as well as damage to DNA. Due to reduction of the protecting ozone layer in the stratosphere, concern about the effects of UV-B radiation on plants has increased. The physiological response to these stresses arises out of changes in cellular gene expression.

The ability to increase plant yield and stress resistance would have many applications in areas such as agriculture, including in the production of ornamental plants, arboriculture, horticulture and forestry. Increasing yield may also find use in the production of algae for use in bioreactors (for the biotechnological production of substances such as pharmaceuticals, antibodies or vaccines, or for the bioconversion of organic waste) and other such areas.

The G1/S transition in the cell cycle of plants and animals is controlled by the RB/E2F/DP pathway. E2F transcription factors form a critical component of this regulatory mechanism (Stals & Inzé, Trends Plant Science 6, 359-364, 2001). The family of E2F transcription factors in Arabidopsis thaliana consists of six members, which may be grouped in two subfamilies (Mariconti et al., J. Biol. Chem. 277, 9911-9919, 2002): on the one hand E2Fa, E2Fb and E2Fc, on the other hand E2Fd, E2Fe and E2Ff. E2F members of the first subfamily have one DNA binding domain and require an interacting DP partner to form either a functional transcriptional activator (E2Fa and E2Fb) or repressor (E2Fc). Members of the second subfamily possess 2 E2F-like DNA binding domains and comprise structural characteristics that discriminate them from the three members of the first subgroup. AtE2Fd and AtE2Fe have a nuclear localisation signal (NLS) in the C-terminal part of the protein, which is sufficient for DNA binding without the need of a DP partner, but AtE2Ff lacks a NLS. Experimental data suggested that the E2F members of the second subfamily have no activation domains and were postulated to interfere with the activity of the other AtE2F proteins (Mariconti et al., 2002).

In Arabidopsis, AtE2Fe (also known as DEL1 or E2L3) is expressed at the beginning of the G₁/S transition and at the S/G₂ transition. The protein is capable of binding E2F sites without a DP partner. A study by Vlieghe et al. (Current Biology 15, 59-63, 2005) demonstrated that AtE2Fe may be involved in controlling the endocycle. It was shown that in a DEL1 mutant (del1-1, with a T-DNA insertion between the two DNA binding domains) ploidy levels were increased, whereas DEL1 overexpressing plants had reduced endoreduplication. The changes in ploidy levels correlated with altered expression levels for a number of E2F target genes involved in DNA replication. DEL1 transcripts were detected in dividing cells but were absent in endocycling cells, which suggested that DEL1 may be involved in repressing the endocycle in mitotically active cells. Barow M. and Meister A. (2003) Plant, Cell and Environment 26, 571-584 suggest that endopolyploid plant species have advantages when growing for example at higher latitudes. The control of the endocycle is a complex process in plants and many genes are involved.

In the present invention it has surprisingly been found that decreasing the level of activity of a DEL1 polypeptide gives plants having increased yield and/or increased stress resistance relative to control plants. Preferably the decreased level of activity is obtained by modulating expression in a plant of a nucleic acid encoding a fragment of a DEL1 polypeptide or by downregulating the expression of a DEL1 gene or a variant thereof. Preferably, the DELL polypeptide is of plant origin.

According to one embodiment of the present invention, there is provided a method for increasing plant yield and/or increasing stress resistance, comprising decreasing the level of activity of a DEL1 polypeptide, preferably by modulating expression in a plant of a nucleic acid encoding a fragment of a DEL1 polypeptide, or by downregulating expression of a DEL1 gene or a variant thereof.

Any reference hereinafter to a “protein useful in the methods of the invention” is taken to mean a DEL1 polypeptide as defined herein. Any reference hereinafter to a “nucleic acid useful in the methods of the invention” is taken to mean a nucleic acid capable of encoding such a DEL1 polypeptide. The terms “polypeptide” and “protein” are used interchangeably herein and refer to amino acids in a polymeric form of any length. The terms “polynucleotide(s)”, “nucleic acid sequence(s)”, “nucleotide sequence(s)” “gene(s)” are used interchangeably herein and refer to nucleotides, either ribonucleotides or deoxyribonucleotides or a combination of both, in a polymeric form of any length.

The choice of suitable control plants is a routine part of an experimental setup and may include corresponding wild type plants or corresponding plants without the gene of interest. The control plant is typically of the same plant species or even of the same variety as the plant to be assessed. The control plant may also be a nullizygote of the plant to be assessed. A “control plant” as used herein refers not only to whole plants, but also to plant parts, including seeds and seed parts.

Advantageously, performance of the methods according to the present invention results in plants having increased yield, particularly increased seed yield and/or increased biomass, relative to control plants.

The term “yield” in general means a measurable produce of economic value, necessarily related to a specified crop, to an area, and to a period of time. Individual plant parts directly contribute to yield based on their number, size and/or weight, whereas the actual yield is the yield per acre for a crop and year, which is determined by dividing total production (includes both harvested and appraised production) by planted acres.

The terms “increase”, “improving” or “improve” are interchangeable and shall mean in the sense of the application at least a 5%, 6%, 7%, 8%, 9% or 10%, preferably at least 15% or 20%, more preferably 25%, 30%, 35% or 40% more yield and/or growth in comparison to the wild type plant as defined herein.

The term “increased yield” as defined herein is taken to mean an increase in any one or more of the following, each relative to control plants: (i) increased biomass (weight) of one or more parts of a plant, particularly aboveground (harvestable) parts, increased root biomass or increased biomass of any other harvestable part; (ii) increased total seed yield, which includes an increase in seed biomass (seed weight) and which may be an increase in the seed weight per plant or on an individual seed basis; (iii) increased number of flowers per plant; (iv) increased number of (filled) seeds; (v) increased seed filling rate (which is expressed as the ratio between the number of filled seeds divided by the total number of seeds); (vi) increased seed size, which may also influence the composition of seeds; (vii) increased seed volume, which may also influence the composition of seeds (including oil, protein and carbohydrate total content and composition); (viii) increased individual seed area; (ix) increased individual seed length and/or seed perimeter; (x) increased harvest index, which is expressed as a ratio of the yield of harvestable parts, such as seeds, over the total biomass; and (xi) increased thousand kernel weight (TKW), which is extrapolated from the number of filled seeds counted and their total weight. An increased TKW may result from an increased seed size and/or seed weight. An increased TKW may also result from an increase in embryo size and/or endosperm size. An increase in yield may also result in modified architecture, or may occur as a result of modified architecture.

Taking corn as an example, a yield increase may be manifested as one or more of the following: increase in the number of plants established per hectare or acre, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per hectare or acre, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

According to a preferred feature, performance of the methods of the invention result in plants having increased yield, particularly increased biomass and/or seed yield. Therefore, according to the present invention, there is provided a method for increasing plant yield, which method comprises decreasing the level of activity of a DEL1 polypeptide, preferably by modulating expression in a plant of a nucleic acid encoding a fragment of a DEL1 polypeptide or by downregulating expression of a DEL1 gene or a variant thereof.

Since the transgenic plants according to the present invention have increased yield, it is likely that these plants exhibit an increased growth rate (during at least part of their life cycle), relative to the growth rate of control plants at a corresponding stage in their life cycle. The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of rice plants followed by, for example, the sowing and optional harvesting of soy bean, potato or any other suitable plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

Performance of the methods of the invention gives plants having an increased growth rate. Therefore, according to the present invention, there is provided a method for increasing yield in plants, which method comprises decreasing the level of activity of a DEL1 polypeptide, preferably by modulating expression in a plant of a nucleic acid encoding a fragment of a DEL1 polypeptide or by downregulating expression of a DEL1 gene or a variant thereof.

Advantageously, performance of the methods according to the present invention results in plants having increased stress resistance, relative to control plants.

Transgenic plants obtained by the method have increased stress resistance; i.e. are capable of growing normally under environmental conditions in which control plants show reduced growth, metabolism, viability, productivity and/or male or female sterility. An increase in yield and/or growth rate occurs whether the plant is under non-stress conditions or whether the plant is exposed to various stresses compared to control plants. The term tolerance as used herein encompasses protection against stress ranging from a delay to substantially a complete inhibition of alteration in cellular metabolism, reduced cell growth and/or cell death caused by stress conditions. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the typical stresses to which a plant may be exposed. These stresses may be the everyday biotic and/or abiotic (environmental) stresses to which a plant is exposed. Typical abiotic or environmental stresses include temperature stresses caused by atypical hot or cold/freezing temperatures; salt stress; water stress (drought or excess water), UV radiation stress. Sunlight does not only contain radiation of the right wavelengths for photosynthesis: radiation of shorter wavelengths, such as ultraviolet-B radiation (UV-B, 280-320 nm) is also present. UV-B is damaging to living organisms since cellular components such as proteins and nucleic acids absorb this energy-rich radiation. Many studies have shown deleterious UV-B effects such as reduced photosynthesis, biomass reduction, decreased protein synthesis and impaired chloroplast function, as well as damage to DNA. In addition, the expression of many genes is changed by UV-B irradiation: e.g., transcription of defence genes is increased, whereas mRNA levels for chloroplastic genes decline. Chemicals may also cause abiotic stresses. Biotic stresses are typically those stresses caused by pathogens, such as bacteria, viruses, fungi and insects. The term “non-stress” conditions as used herein are those environmental conditions that do not significantly go beyond the normal climatic and other abiotic stress conditions that plants may encounter and that allow optimal growth of plants. Persons skilled in the art are aware of normal soil conditions and climatic conditions for a given geographic location.

According to another preferred feature, performance of the methods of the invention result in plants having increased stress resistance. Therefore, according to the present invention, there is provided a method for increasing stress resistance of a plant, which method comprises decreasing the level of activity of a DEL1 polypeptide, preferably by modulating expression in a plant of a nucleic acid encoding a fragment of a DEL1 polypeptide or by down-regulating expression of a DEL1 gene or a variant thereof.

The abovementioned growth characteristics may advantageously be modified in any plant.

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprise the gene/nucleic acid of interest.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agropyron spp., Allium spp., Amaranthus spp., Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Capsicum spp., Carex elata, Carica papaya, Carissa macrocarpa, Carya spp., Carthamus tinctorius, Castanea spp., Cichorium endivia, Cinnamomum spp., Citrullus lanatus, Citrus spp., Cocos spp., Coffea spp., Colocasia esculenta, Cola spp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eriobotrya japonica, Eugenia uniflora, Fagopyrum spp., Fagus spp., Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Passiflora edulis, Pastinaca sativa, Persea spp., Petroselinum crispum, Phaseolus spp., Phoenix spp., Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Triticosecale rimpaui, Triticum spp. (e.g. Triticum aestivum, Triticum durum, Triticum turgidum, Triticum hybernum, Triticum macha, Triticum sativum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

According to a preferred embodiment of the present invention, the plant is a crop plant. Examples of crop plants include soybean, sunflower, canola, alfalfa, rapeseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, sorghum and oats.

The term “DEL1 polypeptide” as defined herein refers to an E2F-like protein with two DNA binding domains of the winged-helix type (E2F-TDP domain, Pfam PF02319, Interpro IPR003316), but lacking a dimerisation domain; see for example FIG. 1. Preferably, the DELL polypeptide comprises the signature sequence 1 (SEQ ID NO: 9)
(Y/T/P) (S/D) RK (Q/D) KSL (G/W) (L/T) LC (T/E/Q/S) (N/R/K) F (L/V) (A/S/T/R) (L/I/R) Y (N/G/P/D)

Further preferably, the signature sequence 1 is
(Y/T/P) (S/D) RK (Q/D) KSL (G/W) (L/T) LC (T/E/Q/S) (N/R/K) F (L/V) (A/S/T) (L/I/R) Y (N/G/ P/D)

More preferably, the signature sequence 1 is
(Y/T) (S/D) RK (Q/D) KSL (G/W) (L/T) LC (T/E/S) (N/R) F (L/V) (A/S/T) (L/I) Y (N/G/D)

Most preferably, the signature sequence 1 is YSRKQKSLGLLCTNFLALYN

Alternative signature sequences for characterising a DEL1 polypeptide include: signature sequence 2 (SEQ ID NO: 10):
(G/S) LD (D/E) (A/V)A(S/A/T/V/R/K) (K/R/S)LGvE;
   signature sequence 3 (SEQ ID NO: 11):
R (R/K) (E/D) KSL (G/A/R) (I/L) (L/M) (T/S) (Q/K) (K/N) F (I/V) (K/Q/M) LF (I/V/L/T) (C/A/V/M/N/T) (S/E/T/M);
   signature sequence 4 (SEQ ID NO: 12):
(I/V/L) (S/T) L (D/E) (D/T/V/E) AA (K/R) (L/I/C/R) (L/I) (L/M/I) (G/E);
   and signature sequence 5 (SEQ ID NO: 13):
(T/A) K (V/I) RRLYDIAN (V/I) L (S/C/T) S (M/L) (N/R/A/H/Q) (L/F) I (E/R/K/D) K (T/V/I) (H/Q/T) (T/V/Q/H) (L/G/A/T/V/P) (D/E) (S/T/E) R (K/G) (P/R) (A/K) (F/P) (K/L/A/R) (W/F) (L/K).

Preferably, signature sequence 2 is (G/S) LDDAA (S/A/T/R/K) (K/R) LGVE, most preferably signature sequence 2 is GLDDAASKLGvE.

Furthermore, signature sequence 3 is preferably
R(R/K) (E/D) KSL (G/A/R) (I/L) (L/M) (T/S) Q (K/N) F (I/V) (K/Q/M) LF (I/V/L/T) (C/A/V/M/N/T) (S/E/M),
   more preferably, signature sequence 3 is RREKSLGLLTQNFIKLFICS.
R (R/K) EKSL (G/A) LL (T/S) Q (K/N) F (I/V) (K/Q) LF (I/V/L/T) (C/A/M/N/T) (S/M),
   most preferably signature sequence 3 is RREKSLGLLTQNFIKLFICS.

Signature sequence 4 is preferably
(I/V) (S/T) L (D/E) (D/T/V/E) AA (K/R) (L/I) (L/I) (L/M/I) (G/E)

More preferably, signature sequence 4 is
(I/V)SL(D/E) (D/T/E)AA(K/R) (L/I) (L/I) (L/M)G, most preferably signature sequence 4 is ISLDDAAKLLLG.

Signature sequence 5 is preferably
(T/A) K (V/I) RRLYDIAN (V/I) L (S/C/T) S (M/L) (N/R/A) (L/F) I (E/R/K/D) K (T/V/I) (H/Q) (T/V/Q) (L/G/A/T) (D/E) (S/T/E) R (K/G) (P/R) (A/K) (F/P) (K/L/A/R) (W/F) (L/K)

More preferably signature sequence 5 is
(T/A) K (V/I) RRLYDIAN (V/I) L (S/C/T) S (M/L) (N/R) (L/F) I (E/R/D) K (T/V/I) (H/Q) (T/V/Q) (L/G/A/T) (D/E) (S/T) RKPAF (K/L/R)WL, most preferably signature sequence 5 is TKVRRLYDIANVLSSMNLIEKTHTLDSRKPAFKWL.

Most preferably, the DEL1 protein is as represented by SEQ ID NO: 8.

The proteins of the invention are identifiable by the presence of the E2F_TDP domain domain(s) (shown in FIG. 1). The term “domain” refers to a set of amino acids conserved at specific positions along an alignment of sequences of evolutionarily related proteins. While amino acids at other positions can vary between homologues, amino acids that are highly conserved at specific positions indicate amino acids that are essential in the structure, stability or activity of a protein. Identified by their high degree of conservation in aligned sequences of a family of protein homologues, they can be used as identifiers to determine if any polypeptide in question belongs to a previously identified polypeptide family (in this case, the proteins useful in the methods of the invention and nucleic acids encoding the same as defined herein).

The term “motif” or “consensus sequence” or “signature sequence” refers to a short conserved region in the sequence of evolutionarily related proteins. Motifs are frequently highly conserved parts of domains, but may also include only part of the domain, or be located outside of conserved domain (if all of the amino acids of the motif fall outside of a defined domain). A person skilled in the art will know that in the signature sequences, on positions where 3 or more amino acids are possible, the sequence is not very conserved and that mismatches may occur on these positions.

The term “DEL1 polypeptide” also encompasses homologues of SEQ ID NO: 8. By aligning other protein sequences with SEQ ID NO: 8, the corresponding signature sequences detailed above may easily be identified. In this way, DEL1 polypeptides or homologues thereof may readily be identified, using routine techniques well known in the art, such as by sequence alignment. Specialist databases also exist for the identification of domains, for example, SMART (Schultz et al. (1998) Proc. Natl. Acad. Sci. USA 95, 5857-5864; Letunic et al. (2002) Nucleic Acids Res 30, 242-244, InterPro (Mulder et al., (2003) Nucl. Acids. Res. 31, 315-318, Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAlPress, Menlo Park; Hulo et al., Nucl. Acids. Res. 32:D134-D137, (2004), or Pfam (Bateman et al., Nucleic Acids Research 30(1): 276-280 (2002). A set of tools for in silico analysis of protein sequences is available on the ExPASY proteomics server (hosted by the Swiss Institute of Bioinformatics (Gasteiger et al., ExPASy: the proteomics server for in-depth protein knowledge and analysis, Nucleic Acids Res. 31:3784-3788 (2003)).

Domains may also be identified using routine techniques, such as by sequence alignment. Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains (such as the E2F_TDP domain, or one of the motifs defined above) may be used as well. The sequence identity values, which are indicated below in Example 3 as a percentage were determined over the entire nucleic acid or amino acid sequence, and/or over selected domains or conserved motif(s), using the programs mentioned above using the default parameters.

Examples of DEL1 polypeptides or homologues are provided in Example 1.

An assay may be carried out to determine DEL1 activity. Examples of assays for determining DELL activity are provided in Example 6.

The sequence given in SEQ ID NO: 21 represents a hitherto unknown DEL1 protein. There is therefore provided an isolated DEL1 polypeptide selected from the group consisting of:
(a) a polypeptide as given in SEQ ID NO 21,
(b) a polypeptide with an amino acid sequence which has at least 60% sequence identity, preferably 70% sequence identity, more preferably 80% or 90% sequence identity, most preferably 95%, 96%, 97%, 98% or 99% sequence identity to the amino acid sequence as given in SEQ ID NO 21,
(c) a homologue, a derivative, and/or functional fragment of a protein as defined in (a) or (b).

It is to be understood that sequences falling under the definition of “DEL1 polypeptide or homologue thereof” are not to be limited to the sequences represented by SEQ ID NO: 8, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29 or SEQ ID NO: 31, but that any polypeptide comprising two DNA binding domains of the winged-helix type (E2F_TDP domain) as described by Pfam record PF02319 or Interpro record IPR003316 but lacking a dimerisation domain and/or comprising one or more of the signature sequences detailed in SEQ ID NO: 9, 10, 11, 12 and 13 may be suitable for use in the methods of the invention.

Also useful in the methods of the invention are homologues of any one of the amino acid sequences given in table A of Example 1. “Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

A deletion refers to removal of one or more amino acids from a protein.

An insertion refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag•100 epitope, c-myc epitope, FLAG®-epitope, lacZ, CMP (calmodulin-binding peptide), HA epitope, protein C epitope and VSV epitope.

A substitution refers to replacement of amino acids of the protein with other amino acids having similar properties. Amino acid substitutions are typically of single residues, but may be clustered depending upon functional constraints placed upon the polypeptide; insertions will usually be of the order of about 1 to 10 amino acid residues. Preferably, amino acid substitutions comprise conservative amino acid substitutions. To produce such homologues, amino acids of the protein may be replaced by other amino acids having similar properties (such as similar hydrophobicity, hydrophilicity, antigenicity, propensity to form or break α-helical structures or β-sheet structures). Conservative substitution tables are well known in the art (see for example Creighton (1984) Proteins. W.H. Freeman and Company and Table 1 below).

TABLE 1
Examples of conserved amino acid substitutions

| Residue | Conservative Substitutions |
|---|---|
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

Amino acid substitutions, deletions and/or insertions may readily be made using peptide synthetic techniques well known in the art, such as solid phase peptide synthesis and the like, or by recombinant DNA manipulations. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

Also useful in the methods of the invention are derivatives of any one of the polypeptides given in table A of Example 1 or orthologues or paralogues of any of the aforementioned SEQ ID Nos. “Derivatives” include peptides, oligopeptides, polypeptides which may, compared to the amino acid sequence of the naturally-occurring form of the protein, such as the one presented in SEQ ID NO: 8, comprise substitutions of amino acids with non-naturally occurring amino acid residues, or additions of non-naturally occurring amino acid residues. Derivatives of the polypeptides given in table A of Example 1 are further examples which may be suitable for use in the methods of the invention. Derivatives useful in the methods of the present invention preferably have similar biological and functional activity as the unmodified protein from which they are derived.

“Derivatives” of a protein also encompass peptides, oligopeptides, polypeptides which comprise naturally occurring altered (glycosylated, acylated, prenylated, phosphorylated, myristoylated, sulphated etc) or non-naturally altered amino acid residues compared to the amino acid sequence of a naturally-occurring form of the polypeptide. A derivative may also comprise one or more non-amino acid substituents or additions compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein.

It is envisaged that decrease of DEL1 activity may also be obtained by modifying the DNA binding domain(s) in a DEL1 protein by deleting, adding or substituting amino acids as described above. Such modified DEL1 proteins may also be useful in the methods of the present invention. Also derivatives of DEL1 proteins, wherein the DNA binding domain(s) is or are modified are envisaged to be useful in the methods of the present invention.

Encompassed by the term “homologues” are orthologous sequences and paralogous sequences, two special forms of homology which encompass evolutionary concepts used to describe ancestral relationships of genes.

The term “paralogous” relates to gene-duplications within the genome of a species leading to paralogous genes. Paralogues of DEL1 may easily be identified by performing a BLAST analysis against a set of sequences from the same species as the query sequence.

Orthologues and paralogues may easily be found by performing a so-called reciprocal blast search. Typically, this involves a first BLAST involving BLASTing a query sequence (for example using any of the sequences listed in table A of Example 1) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 7 or SEQ ID NO: 8, the second BLAST would therefore be against Arabidopsis thaliana sequences). The results of the first and second BLASTs are then compared. A paralogue is identified if a high-ranking hit from the first blast is from the same species as from which the query sequence is derived, a BLAST back then ideally results in the query sequence as highest hit; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues.

Table A of Example 1 gives examples of orthologues and paralogues of the DEL1 protein represented by SEQ ID NO 8. Further orthologues and paralogues may readily be identified using the BLAST procedure described above.

A DEL1 polypeptide is encoded by a DEL1 nucleic acid/gene. Therefore the term “DEL1 nucleic acid/gene” as defined herein is any nucleic acid/gene encoding a DEL1 polypeptide as defined above.

Examples of DEL1 nucleic acids include but are not limited to those represented in Table A of Example 1.

The sequence represented by SEQ ID NO: 20 was hitherto unknown. There is therefore provided an isolated nucleic acid sequence comprising:
(i) a nucleic acid sequence represented by SEQ ID NO: 20, or the complement strand thereof;
(ii) a nucleic acid sequence encoding an amino acid sequence as defined above in (a) to (c);
(iii) a nucleic acid sequence capable of hybridising (preferably under stringent conditions) with a nucleic acid sequence of (i) or (ii) above, which hybridising sequence preferably encodes a protein having yield and/or stress resistance increasing activity;
(iv) a nucleic acid which is an allelic variant to the nucleic acid sequences according to (i) to (iii);
(v) a nucleic acid which is a splice variant to the nucleic acid sequences according to (i) to (iii);
(vi) a nucleic acid sequence which has in increasing order of preference 60%, 70%, 75%, 80%, 85%, 90%, 95%, 96%, 97%, 98% or 99% sequence identity to SEQ ID NO: 20 or the complement thereof; and
(vii) a portion of a nucleic acid sequence according to any of (i) to (vi) above, which portion preferably encodes a protein having yield and/or stress resistance increasing activity.

Nucleic acids encoding DEL1 proteins useful in the methods of the invention need not be full-length nucleic acids, since performance of the methods of the invention does not rely on the use of full-length nucleic acid sequences. Examples of DEL1 nucleic acids suitable for use in performing the methods of the invention include the nucleic acid sequences given in table A of Example 1, but are not limited to those sequences. Nucleic acid variants may also be useful in practising the methods of the invention. Examples of such nucleic acid variants include portions of nucleic acids encoding a protein useful in the methods of the invention, nucleic acids hybridising to nucleic acids encoding a protein useful in the methods of the invention, splice variants of nucleic acids encoding a protein useful in the methods of the invention, allelic variants of nucleic acids encoding a protein useful in the methods of the invention and variants of nucleic acids encoding a protein useful in the methods of the invention that are obtained by gene shuffling. The terms portion, hybridising sequence, splice variant, allelic variant and gene shuffling will now be described.

According to the present invention, there is provided a method for improving growth characteristics of plants, comprising introducing and expressing in a plant a portion of any one of the nucleic acid sequences given in table A of Example 1, or a portion of a nucleic acid encoding an orthologue, paralogue or homologue of any of the amino acid sequences given in table A of Example 1.

The term portion as defined herein refers to a piece of a DNA encoding a DEL1 polypeptide, which piece encodes a polypeptide that lacks at least part of one of the DNA binding domains, but comprises at least the part located C-terminally of the DNA binding domains, and optionally also one or more of the signature sequences defined above. A portion may be prepared, for example, by making one or more deletions to a DEL1 nucleic acid. The portions may be used in isolated form or they may be fused to other coding (or non coding) sequences in order to, for example, produce a protein that combines several activities. When fused to other coding sequences, the resulting polypeptide produced upon translation may be bigger than that predicted for the DEL1 fragment. Preferably, the portion is a portion of a nucleic acid as represented by any one of the sequences listed in Table A of Example 1. More preferably, the portion of a nucleic acid encodes a DEL1 polypeptide that comprises at least the part located C-terminally of the DNA binding domains, and optionally also one or more of the signature sequences defined above, but that lacks at least part of a DNA binding domain or that lacks one or both DNA binding that domains, such deletion resulting in the loss of DNA-binding activity. Most preferably the portion of a nucleic acid is as represented by SEQ ID NO: 1 or encodes a polypeptide represented by SEQ ID NO: 2.

The term “fragment of a DEL1 polypeptide” as used in this invention (hereafter named DEL1f) refers to a DEL1 protein as defined above in which at least part of a DNA binding domain is deleted or that lacks one or both DNA binding domains, but that still comprises the part located C-terminally of the DNA binding domains, and that optionally also comprises on or more of the signature sequences as defined above, such deletion resulting in the loss of DNA-binding activity. Preferably, DEL1f lacks part of the first DNA binding domain, most preferably, DEL1f is as represented by SEQ ID NO: 2.

Another variant of a DEL1 nucleic acid/gene is a nucleic acid capable of hybridising under reduced stringency conditions, preferably under stringent conditions, with a DEL1 nucleic acid/gene as hereinbefore defined, which hybridising sequence encodes a polypeptide comprising at least the sequence located C-terminally of the DNA-binding domains, and preferably also one or more of the signature sequences defined above. The hybridising sequence is typically at least 600 consecutive nucleotides in length, preferably at 800 consecutive nucleotides in length, more preferably at least 1000 consecutive nucleotides in length and most preferably at least 1200 consecutive nucleotides in length, the consecutive nucleotides being of any one of the nucleic acid sequences given in table A of Example 1. Preferably, the hybridising sequence is one that is capable of hybridising to a nucleic acid encoding proteins as represented in Table A of Example 1, or to a portion of any of the aforementioned sequences. Most preferably the hybridising sequence is capable of hybridising to SEQ ID NO: 1 or SEQ ID NO: 7.

The term “hybridisation” as defined herein is a process wherein substantially homologous complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point I for the specific sequence at a defined ionic strength and pH. Medium stringency conditions are when the temperature is 20° C. below Tₘ, and high stringency conditions are when the temperature is 10° C. below Tₘ. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore medium stringency hybridisation conditions may sometimes be needed to identify such nucleic acid molecules.

The Tₘ is the temperature under defined ionic strength and pH, at which 50% of the target sequence hybridises to a perfectly matched probe. The Tₘ is dependent upon the solution conditions and the base composition and length of the probe. For example, longer sequences hybridise specifically at higher temperatures. The maximum rate of hybridisation is obtained from about 16° C. up to 32° C. below Tₘ. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tₘ decreases about 1° C. per % base mismatch. The Tₘ may be calculated using the following equations, depending on the types of hybrids:
(i) DNA-DNA hybrids (Meinkoth and Wahl, Anal. Biochem., 138: 267-284, 1984): Tₘ=81.5° C.+16.6×log₁₀[Na⁺]^{a}+0.41×%[G/C^{b}]−500×[L^{c}]⁻¹−0.61x% formamide
(ii) DNA-RNA or RNA-RNA hybrids: Tₘ=79.8+18.5 (log₁₀[Na⁺]^{a})+0.58 (% G/C^{b})+11.8 (% G/C^{b})²−820/L^{c}
(iii) oligo-DNA or oligo-RNAd hybrids:
   For <20 nucleotides: Tₘ=2 (Iₙ)
   For 20-35 nucleotides: Tₘ=22+1.46 (Iₙ)
^{a} or for other monovalent cation, but only accurate in the 0.01-0.4 M range.
^{b} only accurate for % GC in the 30% to 75% range.
^{c} L=length of duplex in base pairs.
^{d} Oligo, oligonucleotide; Iₙ, effective length of primer=2×(no. of G/C)+(no. of A/T).

Non-specific binding may be controlled using any one of a number of known techniques such as, for example, blocking the membrane with protein containing solutions, additions of heterologous RNA, DNA, and SDS to the hybridisation buffer, and treatment with Rnase. For non-homologous probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCl and 15 mM sodium citrate; the hybridisations and washes may additionally include 5×Denhardt's reagent, 0.5-1.0% SDS, 100 μg/ml denatured, fragmented salmon sperm DNA, 0.5% sodium pyrophosphate.

For the purposes of defining the level of stringency, reference can be made to Sambrook et al. (2001) Molecular Cloning: a laboratory manual, 3^{rd} Edition Cold Spring Harbor Laboratory Press, CSH, New York or to Current Protocols in Molecular Biology, John Wiley & Sons, N.Y. (1989 and yearly updates).

Another nucleic acid variant useful in the methods of the invention is a splice variant encoding a DEL1 polypeptide as defined hereinabove. The term “splice variant” as used herein encompasses variants of a nucleic acid sequence in which selected introns and/or exons have been excised, replaced or added, or in which introns have been shortened or lengthened. Such variants will be ones in which the biological activity of the protein is substantially retained, which may be achieved by selectively retaining functional segments of the protein. Such splice variants may be found in nature or may be manmade. Methods for making such splice variants are known in the art (see for example Foissac and Schiex, BMC Bioinformatics. 2005; 6: 25). Preferred splice variants are splice variants of a nucleic acid encoding a DELL polypeptide as defined above. Further preferred are splice variants of nucleic acids encoding orthologues or paralogues of the polypeptide represented by SEQ ID NO: 8, such as the polypeptides listed in Table A of Example 1. Most preferred is the splice variant represented by SEQ ID NO: 7.

Another nucleic acid variant useful in performing the methods of the invention is an allelic variant of a nucleic acid encoding a DEL1 polypeptide as defined hereinabove. Alleles or allelic variants are alternative forms of a given gene, located at the same chromosomal position. Allelic variants exist in nature, and encompassed within the methods of the present invention is the use of these natural alleles. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms. Preferred allelic variants are allelic variants of nucleic acids encoding orthologues and paralogues of the polypeptide of SEQ ID NO: 8, such as the polypeptides listed in Table A of Example 1. Most preferred is the allelic variant represented by SEQ ID NO: 7.

A further nucleic acid variant useful in the methods of the invention is a nucleic acid variant obtained by gene shuffling. Gene shuffling or directed evolution may also be used to generate variants of nucleic acids encoding DEL1 or DEL1f proteins as defined above. This consists of iterations of DNA shuffling followed by appropriate screening and/or selection to generate variants of DEL1 nucleic acids or portions thereof encoding DEL1 polypeptides or portions thereof having an modified biological activity (Castle et al., (2004) Science 304(5674): 1151-4; U.S. Pat. Nos. 5,811,238 and 6,395,547). Preferably, the variant nucleic acid obtained by gene shuffling encodes a DEL1 or DEL1f polypeptide comprising any one or more of the motifs or domains as defined herein.

Furthermore, nucleic acid variants, including nucleic acids encoding a DEL1f polypeptide, may also be obtained by site-directed mutagenesis. Several methods are available to achieve site-directed mutagenesis, the most common being PCR based methods (Current Protocols in Molecular Biology. Wiley Eds.).

The DEL1 nucleic acid or variant thereof may be derived from any natural or artificial source. The nucleic acid/gene or variant thereof may be isolated from a microbial source, such as yeast or fungi, or from a plant, algae or animal (including human) source. This nucleic acid may be modified from its native form in composition and/or genomic environment through deliberate human manipulation. The nucleic acid is preferably of plant origin, whether from the same plant species (for example to the one in which it is to be introduced) or whether from a different plant species. The nucleic acid may be isolated from a dicotyledonous species, preferably from the family Brassicaceae, further preferably from Arabidopsis thaliana. More preferably, the DEL1 nucleic acid is isolated from Arabidopsis thaliana and is represented by SEQ ID NO: 7, and the DEL1 amino acid sequence is as represented by SEQ ID NO: 8, whereas DEL1f is represented by SEQ ID NO: 1, and the DEL1f amino acid sequence is as represented by SEQ ID NO: 2.

Any reference herein to a DEL1 or DEL1f protein is therefore taken to mean a DEL1 or DEL1f protein as defined above. Any nucleic acid encoding such a DEL1 or DEL1f protein is suitable for use in performing the methods of the invention.

The expression of a nucleic acid encoding a DEL1 polypeptide or a homologue or fragment thereof may be modulated by introducing a genetic modification (preferably in the locus of a DEL1 gene). The locus of a gene as defined herein is taken to mean a genomic region, which includes the gene of interest and 10 kb up- or down stream of the coding region.

The genetic modification may be introduced, for example, by any one (or more) of the following methods: T-DNA activation, TILLING, site-directed mutagenesis, transposon mutagenesis, directed evolution and homologous recombination or by introducing and expressing in a plant a nucleic acid encoding a DEL1 polypeptide or a fragment thereof. Following introduction of the genetic modification, there follows a step of selecting for modified expression of a nucleic acid encoding a DEL1 polypeptide or a fragment thereof, which modification in expression gives plants having increased yield and/or increased stress resistance.

T-DNA activation tagging (Hayashi et al. Science (1992) 1350-1353) involves insertion of T-DNA, usually containing a promoter (may also be a translation enhancer or an intron), in the genomic region of the gene of interest or 10 kb up- or down stream of the coding region of a gene in a configuration such that the promoter directs expression of the targeted gene. Typically, regulation of expression of the targeted gene by its natural promoter is disrupted and the gene falls under the control of the newly introduced promoter. The promoter is typically embedded in a T-DNA. This T-DNA is randomly inserted into the plant genome, for example, through Agrobacterium infection and leads to overexpression of genes near the inserted T-DNA. The resulting transgenic plants show dominant phenotypes due to overexpression of genes close to the introduced promoter. The promoter to be introduced may be any promoter capable of directing expression of a gene in the desired organism, in this case a plant. For example, constitutive, tissue-preferred, cell type-preferred and inducible promoters are all suitable for use in T-DNA activation.

A genetic modification may also be introduced in the locus of a DEL1 gene using the technique of TILLING (Targeted Induced Local Lesions In Genomes). This is a mutagenesis technology useful to generate and/or identify, and to eventually isolate mutagenised variants of a DEL1 nucleic acid capable of exhibiting DEL1 or DEL1f activity. TILLING also allows selection of plants carrying such mutant variants. These mutant variants may even exhibit higher DEL1 or DEL1f activity than that exhibited by the gene in its natural form. TILLING combines high-density mutagenesis with high-throughput screening methods. The steps typically followed in TILLING are: (a) EMS mutagenesis (Redei GP and Koncz C (1992) In Methods in Arabidopsis Research, Koncz C, Chua N H, Schell J, eds. Singapore, World Scientific Publishing Co, pp. 16-82; Feldmann et al., (1994) In Meyerowitz E M, Somerville C R, eds, Arabidopsis. Cold Spring Harbor Laboratory Press, Cold Spring Harbor, N.Y., pp 137-172; Lightner J and Caspar T (1998) In J Martinez-Zapater, J Salinas, eds, Methods on Molecular Biology, Vol. 82. Humana Press, Totowa, N.J., pp 91-104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (McCallum et al., (2000) Nat Biotechnol 18: 455-457; reviewed by Stemple (2004) Nat Rev Genet. 5(2): 145-50).

Transposon mutagenesis is a mutagenesis technique based on the insertion of transposons in genes, which frequently results in gene-knockout. The technique has been used for several plant species, including rice (Greco et al., Plant Physiol, 125, 1175-1177, 2001), corn (McCarty et al., Plant J. 44, 52-61, 2005) and Arabidopsis (Parinov and Sundaresan, Curr. Opin. Biotechnol. 11, 157-161, 2000).

T-DNA activation, TILLING, site-directed mutagenesis, transposon mutagenesis and directed evolution are examples of technologies that enable the generation of novel alleles and DEL1 variants, and/or modulation of their expression.

Homologous recombination allows introduction in a genome of a selected nucleic acid at a defined selected position. Homologous recombination is a standard technology used routinely in biological sciences for lower organisms such as yeast or the moss Physcomitrella. Methods for performing homologous recombination in plants have been described not only for model plants (Offring a et al. (1990) EMBO J. 9(10): 3077-84) but also for crop plants, for example rice (Terada et al. (2002) Nat Biotech 20(10): 1030-4; lida and Terada (2004) Curr Opin Biotech 15(2):132-8).

A preferred method for introducing a genetic modification (which in this case need not be in the locus of a DEL1 gene) is to introduce and express in a plant a nucleic acid encoding a DELL polypeptide or a fragment thereof, as defined above. The nucleic acid to be introduced into a plant may be a full-length nucleic acid or may be a portion or a hybridising sequence as hereinbefore defined.

According to a preferred aspect of the present invention, increased expression of the DEL1f nucleic acid or variant thereof is envisaged. Methods for increasing expression of genes or gene products are well documented in the art and include, for example, overexpression driven by appropriate promoters, the use of transcription enhancers or translation enhancers. If for example increased expression of an endogenous gene is desired, isolated nucleic acids which serve as promoter or enhancer elements may be introduced in an appropriate position (typically upstream) of a non-heterologous form of a polynucleotide so as to upregulate expression of a DEL1 nucleic acid or variant thereof. For example, endogenous promoters may be altered in vivo by mutation, deletion, and/or substitution (see, Kmiec, U.S. Pat. No. 5,565,350; Zarling et al., PCT/US93/03868), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide-coding region. The polyadenylation region may be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold, Buchman and Berg, Mol. Cell. Biol. 8:4395-4405 (1988); Callis et al., Genes Dev. 1:1183-1200 (1987). Such intron enhancement of gene expression is typically greatest when placed near the 5′ end of the transcription unit. Use of the maize introns Adh1-S intron 1, 2, and 6, the Bronze-1 intron are known in the art. See generally, The Maize Handbook, Chapter 116, Freeling and Walbot, Eds., Springer, N.Y. (1994).

According to another preferred aspect of the present invention, decreased expression (a reduction or substantial elimination) of a DEL1 nucleic acid sequence is envisaged.

Reference herein to “reduction or substantial elimination” is taken to mean a decrease in endogenous gene expression and/or polypeptide levels and/or polypeptide activity relative to control plants. The reduction or substantial elimination is in increasing order of preference at least 10%, 20%, 30%, 40% or 50%, 60%, 70%, 80%, 85%, 90%, or 95%, 96%, 97%, 98%, 99% or more reduced compared to that of control plants.

Reference herein to an “endogenous” gene not only refers to the gene in question as found in a plant in its natural form (i.e., without there being any human intervention), but also refers to that same gene (or a substantially homologous nucleic acid/gene) in an isolated form subsequently (re)introduced into a plant (a transgene). For example, a transgenic plant containing such a transgene may encounter a substantial reduction of the transgene expression and/or substantial reduction of expression of the endogenous gene.

For the reduction or substantial elimination of expression an endogenous gene in a plant, a sufficient length of substantially contiguous nucleotides of a nucleic acid sequence is required. In order to perform gene silencing, this may be as little as 20, 19, 18, 17, 16, 15, 14, 13, 12, 11, 10 or fewer nucleotides, alternatively this may be as much as the entire gene (including the 5′ and/or 3′ UTR, either in part or in whole). The stretch of substantially contiguous nucleotides may be derived from SEQ ID NO: 1 or SEQ ID NO: 7, or from any of the nucleic acid sequences given in Table A of Example 1, or from any nucleic acid capable of encoding an orthologue, paralogue or homologue of any one of the amino acid sequences given in Table A. A nucleic acid sequence encoding a (functional) polypeptide is not a requirement for the various methods discussed herein for the reduction or substantial elimination of expression of an endogenous gene.

This reduction or substantial elimination of expression may be achieved using routine tools and techniques. A preferred method for the reduction or substantial elimination of endogenous gene expression is by introducing and expressing in a plant a genetic construct into which the nucleic acid (in this case a stretch of substantially contiguous nucleotides derived from SEQ ID NO: 1 or SEQ ID NO: 7, or from any of the nucleic acid sequences given in Table A of Example 1, or from any nucleic acid capable of encoding an orthologue, paralogue or homologue of any one of the amino acid sequences given in Table A) is cloned as an inverted repeat (in part or completely), separated by a spacer (non-coding DNA).

In such a preferred method, expression of the endogenous gene is reduced or substantially eliminated through RNA-mediated silencing using an inverted repeat of a nucleic acid or a part thereof (in this case a stretch of substantially contiguous nucleotides derived from SEQ ID NO: 1 or SEQ ID NO: 7, or from any of the nucleic acid sequences given in Table A of Example 1, or from any nucleic acid capable of encoding an orthologue, paralogue or homologue of any one of the amino acid sequences given in Table A), preferably capable of forming a hairpin structure. The inverted repeat is cloned in an expression vector comprising control sequences. A non-coding DNA nucleic acid sequence (a spacer, for example a matrix attachment region fragment (MAR), an intron, a polylinker, etc.) is located between the two inverted nucleic acids forming the inverted repeat. After transcription of the inverted repeat, a chimeric RNA with a self-complementary structure is formed (partial or complete). This double-stranded RNA structure is referred to as the hairpin RNA (hpRNA). The hpRNA is processed by the plant into siRNAs that are incorporated into an RNA-induced silencing complex (RISC). The RISC further cleaves the mRNA transcripts, thereby substantially reducing the number of mRNA transcripts to be translated into polypeptides. For further general details see for example, Grierson et al. (1998) WO 98/53083; Waterhouse et al. (1999) WO 99/53050).

Performance of the methods of the invention does not rely on introducing and expressing in a plant a genetic construct into which the nucleic acid is cloned as an inverted repeat, but any one or more of several well-known “gene silencing” methods may be used to achieve the same effects.

One such method for the reduction of endogenous gene expression is RNA-mediated silencing of gene expression (downregulation). Silencing in this case is triggered in a plant by a double stranded RNA sequence (dsRNA) that is substantially similar to the target endogenous gene. This dsRNA is further processed by the plant into about 20 to about 26 nucleotides called short interfering RNAs (siRNAs). The siRNAs are incorporated into an RNA-induced silencing complex (RISC) that cleaves the mRNA transcript of the endogenous target gene, thereby substantially reducing the number of mRNA transcripts to be translated into a polypeptide. Preferably, the double stranded RNA sequence corresponds to a target gene.

Another example of an RNA silencing method involves the introduction of nucleic acid sequences or parts thereof (in this case a stretch of substantially contiguous nucleotides derived from SEQ ID NO: 1 or SEQ ID NO: 7, or from any of the nucleic acid sequences given in Table A of Example 1, or from any nucleic acid capable of encoding an orthologue, paralogue or homologue of any one of the amino acid sequences given in Table A) in a sense orientation into a plant. “Sense orientation” refers to a DNA sequence that is homologous to an mRNA transcript thereof. Introduced into a plant would therefore be at least one copy of the nucleic acid sequence. The additional nucleic acid sequence will reduce expression of the endogenous gene, giving rise to a phenomenon known as co-suppression. The reduction of gene expression will be more pronounced if several additional copies of a nucleic acid sequence are introduced into the plant, as there is a positive correlation between high transcript levels and the triggering of co-suppression.

Another example of an RNA silencing method involves the use of antisense nucleic acid sequences. An “antisense” nucleic acid sequence comprises a nucleotide sequence that is complementary to a “sense” nucleic acid sequence encoding a protein, i.e. complementary to the coding strand of a double-stranded cDNA molecule or complementary to an mRNA transcript sequence. The antisense nucleic acid sequence is preferably complementary to the endogenous gene to be silenced. The complementarity may be located in the “coding region” and/or in the “non-coding region” of a gene. The term “coding region” refers to a region of the nucleotide sequence comprising codons that are translated into amino acid residues. The term “non-coding region” refers to 5′ and 3′ sequences that flank the coding region that are transcribed but not translated into amino acids (also referred to as 5′ and 3′ untranslated regions).

Antisense nucleic acid sequences can be designed according to the rules of Watson and Crick base pairing. The antisense nucleic acid sequence may be complementary to the entire nucleic acid sequence (in this case a stretch of substantially contiguous nucleotides derived from SEQ ID NO: 1 or SEQ ID NO: 7, or from any of the nucleic acid sequences given in Table A of Example 1, or from any nucleic acid capable of encoding an orthologue, paralogue or homologue of any one of the amino acid sequences given in Table A), but may also be an oligonucleotide that is antisense to only a part of the nucleic acid sequence (including the mRNA 5′ and 3′ UTR). For example, the antisense oligonucleotide sequence may be complementary to the region surrounding the translation start site of an mRNA transcript encoding a polypeptide. The length of a suitable antisense oligonucleotide sequence is known in the art and may start from about 50, 45, 40, 35, 30, 25, 20, 15 or 10 nucleotides in length or less. An antisense nucleic acid sequence according to the invention may be constructed using chemical synthesis and enzymatic ligation reactions using methods known in the art. For example, an antisense nucleic acid sequence (e.g., an antisense oligonucleotide sequence) may be chemically synthesized using naturally occurring nucleotides or variously modified nucleotides designed to increase the biological stability of the molecules or to increase the physical stability of the duplex formed between the antisense and sense nucleic acid sequences, e.g., phosphorothioate derivatives and acridine substituted nucleotides may be used. Examples of modified nucleotides that may be used to generate the antisense nucleic acid sequences are well known in the art. Known nucleotide modifications include methylation, cyclization and ‘caps’ and substitution of one or more of the naturally occurring nucleotides with an analogue such as inosine. Other modifications of nucleotides are well known in the art.

The antisense nucleic acid sequence can be produced biologically using an expression vector into which a nucleic acid sequence has been subcloned in an antisense orientation (i.e., RNA transcribed from the inserted nucleic acid will be of an antisense orientation to a target nucleic acid of interest). Preferably, production of antisense nucleic acid sequences in plants occurs by means of a stably integrated nucleic acid construct comprising a promoter, an operably linked antisense oligonucleotide, and a terminator.

The nucleic acid molecules used for silencing in the methods of the invention (whether introduced into a plant or generated in situ) hybridize with or bind to mRNA transcripts and/or genomic DNA encoding a polypeptide to thereby inhibit expression of the protein, e.g., by inhibiting transcription and/or translation. The hybridization can be by conventional nucleotide complementarity to form a stable duplex, or, for example, in the case of an antisense nucleic acid sequence which binds to DNA duplexes, through specific interactions in the major groove of the double helix. Antisense nucleic acid sequences may be introduced into a plant by transformation or direct injection at a specific tissue site. Alternatively, antisense nucleic acid sequences can be modified to target selected cells and then administered systemically. For example, for systemic administration, antisense nucleic acid sequences can be modified such that they specifically bind to receptors or antigens expressed on a selected cell surface, e.g., by linking the antisense nucleic acid sequence to peptides or antibodies which bind to cell surface receptors or antigens. The antisense nucleic acid sequences can also be delivered to cells using the vectors described herein.

According to a further aspect, the antisense nucleic acid sequence is an a-anomeric nucleic acid sequence. An a-anomeric nucleic acid sequence forms specific double-stranded hybrids with complementary RNA in which, contrary to the usual b-units, the strands run parallel to each other (Gaultier et al. (1987) Nucl Ac Res 15: 6625-6641). The antisense nucleic acid sequence may also comprise a 2′-o-methylribonucleotide (Inoue et al. (1987) Nucl Ac Res 15, 6131-6148) or a chimeric RNA-DNA analogue (Inoue et al. (1987) FEBS Lett. 215, 327-330).

The reduction or substantial elimination of endogenous gene expression may also be performed using ribozymes. Ribozymes are catalytic RNA molecules with ribonuclease activity that are capable of cleaving a single-stranded nucleic acid sequence, such as an mRNA, to which they have a complementary region. Thus, ribozymes (e.g., hammerhead ribozymes (described in Haselhoff and Gerlach (1988) Nature 334, 585-591) can be used to catalytically cleave mRNA transcripts encoding a polypeptide, thereby substantially reducing the number of mRNA transcripts to be translated into a polypeptide. A ribozyme having specificity for a nucleic acid sequence can be designed (see for example: Cech et al. U.S. Pat. No. 4,987,071; and Cech et al. U.S. Pat. No. 5,116,742). Alternatively, mRNA transcripts corresponding to a nucleic acid sequence can be used to select a catalytic RNA having a specific ribonuclease activity from a pool of RNA molecules (Bartel and Szostak (1993) Science 261, 1411-1418). The use of ribozymes for gene silencing in plants is known in the art (e.g., Atkins et al. (1994) WO 94/00012; Lenne et al. (1995) WO 95/03404; Lutziger et al. (2000) WO 00/00619; Prinsen et al. (1997) WO 97/13865 and Scott et al. (1997) WO 97/38116).

Gene silencing may also be achieved by insertion mutagenesis (for example, T-DNA insertion or transposon insertion) or by strategies as described by, among others, Angell and Baulcombe ((1999) Plant J 20(3): 357-62), (Amplicon VIGS WO 98/36083), or Baulcombe (WO 99/15682). Therefore, according to another preferred aspect of the invention, there is provided a method for increasing yield and/or increasing stress resistance of a plant by down-regulating expression of an endogenous DEL1 gene upon insertion mutagenesis.

Gene silencing may also occur if there is a mutation on an endogenous gene and/or a mutation on an isolated gene/nucleic acid subsequently introduced into a plant. The reduction or substantial elimination may be caused by a non-functional polypeptide. For example, a polypeptide may bind to various interacting proteins; one or more mutation(s) and/or truncation(s) may therefore provide for a polypeptide that is still able to bind interacting proteins (such as receptor proteins) but that cannot exhibit its normal function (such as signalling ligand).

A further approach to gene silencing is by targeting nucleic acid sequences complementary to the regulatory region of the gene (e.g., the promoter and/or enhancers) to form triple helical structures that prevent transcription of the gene in target cells. See Helene, C., Anticancer Drug Res. 6, 569-84, 1991; Helene et al., Ann. N.Y. Acad. Sci. 660, 27-36 1992; and Maher, L. J. Bioassays 14, 807-15, 1992.

Other methods, such as the use of antibodies directed to an endogenous polypeptide for inhibiting its function in planta, or interference in the signalling pathway in which a polypeptide is involved, will be well known to the skilled man. In particular, it can be envisaged that manmade molecules may be useful for inhibiting the biological function of a target polypeptide, or for interfering with the signalling pathway in which the target polypeptide is involved.

Alternatively, a screening program may be set up to identify in a plant population natural variants of a gene, which variants encode polypeptides with reduced activity. Such natural variants may also be used for example, to perform homologous recombination.

Artificial and/or natural microRNAs (miRNAs) may be used to knock out gene expression and/or mRNA translation. Endogenous miRNAs are single stranded small RNAs of typically 19-24 nucleotides long. They function primarily to regulate gene expression and/or mRNA translation. Most plant microRNAs (miRNAs) have perfect or near-perfect complementarity with their target sequences. However, there are natural targets with up to five mismatches. They are processed from longer non-coding RNAs with characteristic fold-back structures by double-strand specific Rnases of the Dicer family. Upon processing, they are incorporated in the RNA-induced silencing complex (RISC) by binding to its main component, an Argonaute protein. mRNAs serve as the specificity components of RISC, since they base-pair to target nucleic acids, mostly mRNAs, in the cytoplasm. Subsequent regulatory events include target mRNA cleavage and destruction and/or translational inhibition. Effects of miRNA overexpression are thus often reflected in decreased mRNA levels of target genes.

Artificial microRNAs (amiRNAs), which are typically 21 nucleotides in length, can be genetically engineered specifically to negatively regulate gene expression of single or multiple genes of interest. Determinants of plant microRNA target selection are well known in the art. Empirical parameters for target recognition have been defined and can be used to aid in the design of specific amiRNAs, Schwab R, 2005. Convenient tools for design and generation of amiRNAs and their precursors are also available to the public, Schwab et al., 2006.

For optimal performance, the gene silencing techniques used for reducing expression in a plant of an endogenous gene requires the use of nucleic acid sequences from monocotyledonous plants for transformation of monocotyledonous plants, and from dicotyledonous plants for transformation of dicotyledonous plants. Preferably, a nucleic acid sequence from any given plant species is introduced into that same species. For example, a nucleic acid sequence from rice is transformed into a rice plant. However, it is not an absolute requirement that the nucleic acid sequence to be introduced originates from the same plant species as the plant in which it will be introduced. It is sufficient that there is substantial homology between the endogenous target gene and the nucleic acid to be introduced.

Described above are examples of various methods for the reduction or substantial elimination of expression in a plant of an endogenous gene. A person skilled in the art would readily be able to adapt the aforementioned methods for silencing so as to achieve reduction of expression of an endogenous gene in a whole plant or in parts thereof through the use of an appropriate promoter, for example.

Genetic constructs aimed at silencing gene expression may comprise the DEL1 nucleotide sequence, for example as represented by SEQ ID NO: 7 (or one or more portions thereof) in a sense and/or antisense orientation relative to the promoter sequence. The sense or antisense copies of at least part of the endogenous gene in the form of direct or inverted repeats may be utilised in the methods according to the invention. The growth characteristics of plants, and in particular the yield increase and/or increase in stress resistance, may also be improved by introducing into a plant at least part of an antisense version of a DEL1 nucleotide sequence, for example represented by SEQ ID NO: 7. It should be clear that part of the nucleic acid (a portion) could achieve the desired result. Homologous anti-sense genes are preferred to heterologous anti-sense genes, homologous genes being plant genes from the same plant species in which the silencing construct is introduced, and heterologous genes being genes from related or unrelated plant species.

The invention also provides genetic constructs and vectors to facilitate introduction and/or expression of the nucleotide sequences useful in the methods according to the invention.

Therefore, there is provided a gene construct comprising:
(i) a nucleic acid encoding a DEL1f polypeptide, or variant thereof as defined hereinabove;
(ii) one or more control sequences capable of driving expression of the nucleic acid sequence of (i); and optionally
(iii) a transcription termination sequence.

Constructs useful in the methods according to the present invention may be constructed using recombinant DNA technology well known to persons skilled in the art. The nucleic acid constructs may be inserted into vectors, which may be commercially available, suitable for transforming into plants and suitable for expression of the nucleic acid of interest in the transformed cells.

Plants are transformed with a vector comprising the sequence of interest (i.e., a nucleic acid encoding the DEL1f polypeptide or an equivalent fragment of a DEL1 protein, or a nucleic acid sequence designed for the reduction or substantial elimination of expression of an endogenous DEL1 gene). The skilled artisan is well aware of the genetic elements that must be present on the vector in order to successfully transform, select and propagate host cells containing the sequence of interest. The sequence of interest is operably linked to one or more control sequences (at least to a promoter). The terms “regulatory element”, “control sequence” and “promoter” are all used interchangeably herein and are to be taken in a broad context to refer to regulatory nucleic acid sequences capable of effecting expression of the sequences to which they are ligated. The term “promoter” typically refers to a nucleic acid control sequence located upstream from the transcriptional start of a gene and which is involved in recognising and binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid molecule in a cell, tissue or organ. The term “operably linked” as used herein refers to a functional linkage between the promoter sequence and the gene of interest, such that the promoter sequence is able to initiate transcription of the gene of interest.

Advantageously, any type of promoter may be used to drive expression of the nucleic acid sequence. The term “promoter” refers to a nucleic acid control sequence located upstream from the transcriptional start of a gene and which is involved in recognizing and binding of RNA polymerase and other proteins, thereby directing transcription of an operably linked nucleic acid. A “plant” promoter comprises regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g. from the plant which is transformed with the nucleic acid sequence to be expressed in the inventive process and described herein. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, even promoters from heterologous organisms. For expression in plants, the nucleic acid molecule must, as described above, be linked operably to or comprise a suitable promoter which expresses the gene at the right point in time and with the required spatial expression pattern.

The promoter may be a constitutive promoter, which refers to a promoter that is transcriptionally active during most, but not necessarily all, phases of its growth and development and under most environmental conditions, in at least one cell, tissue or organ. Alternatively, the promoter may be an inducible promoter, i.e. having induced or increased transcription initiation in response to a chemical (for a review see Gatz 1997, Annu. Rev. Plant Physiol. Plant Mol. Biol., 48:89-108), environmental or physical stimulus. Another example of an inducible promoter is a stress-inducible promoter, i.e. a promoter activated when a plant is exposed to various stress conditions, or a pathogen-induced promoter.

Additionally or alternatively, the promoter may be an organ-specific or tissue-specific promoter, i.e. one that is capable of preferentially initiating transcription in certain organs or tissues, such as the leaves, roots, seed tissue etc; or the promoter may be a ubiquitous promoter, which is active in substantially all tissues or cells of an organism, or the promoter may be developmentally regulated, thereby being active during certain developmental stages or in parts of the plant that undergo developmental changes. Promoters able to initiate transcription in certain organs or tissues only are referred to herein as “organ-specific” or “tissue-specific” respectively, similarly, promoters able to initiate transcription in certain cells only are referred to herein as “cell-specific”.

Preferably, the DEL1f nucleic acid (i.e. nucleic acid encoding a DEL1f polypeptide) or variant thereof is operably linked to a seed-specific promoter. A seed-specific promoter is transcriptionally active predominantly in seed tissue, but not necessarily exclusively in seed tissue (in cases of leaky expression). The seed-specific promoter may be active during seed development and/or during germination. Seed-specific promoters are well known in the art. Preferably, the seed-specific promoter is the promoter represented by SEQ ID NO: 5 or a functionally equivalent promoter. It should be clear that the applicability of the present invention is not restricted to the DEL1f nucleic acid represented by SEQ ID NO: 1, nor is the applicability of the invention restricted to expression of a DEL1f nucleic acid when driven by a seed-specific promoter. Examples of other seed-specific promoters which may also be used to drive expression of a DEL1 nucleic acid are shown in Table 2 below. Further examples of seed-specific promoters are given in Qing Qu and Takaiwa (Plant Biotechnol. J. 2, 113-125, 2004), which disclosure is incorporated by reference herein as if fully set forth.

TABLE 2
Examples of seed-specific promoters

| Gene source | Reference |
|---|---|
| seed-specific genes | Simon et al., Plant Mol. Biol. 5: 191, 1985; |
| | Scofield et al., J. Biol. Chem. 262: 12202, 1987.; |
| | Baszczynski et al., Plant Mol. Biol. 14: 633, 1990. |
| Brazil Nut albumin | Pearson et al., Plant Mol. Biol. 18: 235-245, 1992. |
| Legumin | Ellis et al., Plant Mol. Biol. 10: 203-214, 1988. |
| Glutelin (rice) | Takaiwa et al., Mol. Gen. Genet. 208: 15-22, 1986; |
| | Takaiwa et al., FEBS Letts. 221: 43-47, 1987. |
| Zein | Matzke et al Plant Mol Biol, 14(3): 323-32 1990 |
| napA | Stalberg et al, Planta 199: 515-519, 1996. |
| Wheat LMW and HMW | Mol Gen Genet 216: 81-90, 1989; NAR 17: 461-2, 1989 |
| glutenin-1 | |
| wheat SPA | Albani et al, Plant Cell, 9: 171-184, 1997 |
| wheat α, β, γ-gliadins | EMBO J. 3: 1409-15, 1984 |
| barley Itr1 promoter | Diaz et al. (1995) Mol Gen Genet 248(5): 592-8 |
| barley B1, C, D, hordein | Theor Appl Gen 98: 1253-62, 1999; Plant J 4: 343-55, 1993; |
| | Mol Gen Genet 250: 750-60, 1996 |
| barley DOF | Mena et al, The Plant Journal, 116(1): 53-62, 1998 |
| blz2 | EP99106056.7 |
| synthetic promoter | Vicente-Carbajosa et al., Plant J. 13: 629-640, 1998. |
| Rice prolamin NRP33 | Wu et al, Plant Cell Physiology 39(8) 885-889, 1998 |
| rice a-globulin Glb-1 | Wu et al, Plant Cell Physiology 39(8) 885-889, 1998 |
| rice OSH1 | Sato et al, Proc. Natl. Acad. Sci. USA, 93: 8117-8122, 1996 |
| rice α-globulin REB/OHP-1 | Nakase et al. Plant Mol. Biol. 33: 513-522, 1997 |
| rice ADP-glucose | Trans Res 6: 157-68, 1997 |
| pyrophosphorylase | |
| maize ESR gene family | Plant J 12: 235-46, 1997 |
| sorghum α-kafirin | DeRose et al., Plant Mol. Biol 32: 1029-35, 1996 |
| KNOX | Postma-Haarsma et al, Plant Mol. Biol. 39: 257-71, 1999 |
| rice oleosin | Wu et al, J. Biochem. 123: 386, 1998 |
| sunflower oleosin | Cummins et al., Plant Mol. Biol. 19: 873-876, 1992 |
| PRO0117, putative rice 40S | WO 2004/070039 |
| ribosomal protein | |
| PRO0136, rice alanine | unpublished |
| aminotransferase | |
| PRO0147, trypsin inhibitor ITR1 | unpublished |
| (barley) | |
| PRO0151, rice WSI18 | WO 2004/070039 |
| PRO0175, rice RAB21 | WO 2004/070039 |
| PRO005 | WO 2004/070039 |
| PRO0095 | WO 2004/070039 |
| α-amylase (Amy32b) | Lanahan et al, Plant Cell 4: 203-211, 1992; Skriver et al, |
| | Proc Natl Acad Sci USA 88: 7266-7270, 1991 |
| cathepsin β-like gene | Cejudo et al, Plant Mol Biol 20: 849-856, 1992 |
| Barley Ltp2 | Kalla et al., Plant J. 6: 849-60, 1994 |
| Chi26 | Leah et al., Plant J. 4: 579-89, 1994 |
| Maize B-Peru | Selinger et al., Genetics 149; 1125-38, 1998 |

In another embodiment, the DEL1 nucleic acid or variant thereof, is operably linked to a constitutive promoter as defined above. Preferably, the constitutive promoter has a comparable expression pattern as the CaMV 35S promoter, more preferably, the constitutive promoter has the same expression pattern as the CaMV 35S promoter, most preferably, the constitutive promoter is the CaMV 35S promoter. It should be clear that the applicability of the present invention is not restricted to the DEL1f nucleic acid represented by SEQ ID NO: 1, nor is the applicability of the invention restricted to expression of a DEL1f nucleic acid when driven by a constitutive promoter. Examples of other constitutive promoters which may also be used to drive expression of a DEL1 nucleic acid are shown in Table 3 below.

TABLE 3
Examples of constitutive promoters

| Gene Source | Reference |
|---|---|
| Actin | McElroy et al, Plant Cell, 2: 163-171, 1990 |
| CAMV 35S | Odell et al, Nature, 313: 810-812, 1985 |
| CaMV 19S | Nilsson et al., Physiol. Plant. 100: 456-462, 1997 |
| GOS2 | de Pater et al, Plant J Nov; 2(6): 837-44, 1992, |
| | WO 2004/065596 |
| Ubiquitin | Christensen et al, Plant Mol. Biol. 18: 675-689, 1992 |
| Rice cyclophilin | Buchholz et al, Plant Mol Biol. 25(5): 837-43, 1994 |
| Maize H3 histone | Lepetit et al, Mol. Gen. Genet. 231: 276-285, 1992 |
| Alfalfa H3 | Wu et al. Plant Mol. Biol. 11: 641-649, 1988 |
| histone | |
| Actin 2 | An et al, Plant J. 10(1); 107-121, 1996 |
| 34S FMV | Sanger et al., Plant. Mol. Biol., 14, 1990: 433-443 |
| Rubisco small | U.S. Pat. No. 4,962,028 |
| subunit | |
| OCS | Leisner (1988) Proc Natl Acad Sci USA 85(5): 2553 |
| SAD1 | Jain et al., Crop Science, 39 (6), 1999: 1696 |
| SAD2 | Jain et al., Crop Science, 39 (6), 1999: 1696 |
| nos | Shaw et al. (1984) Nucleic Acids Res. 12(20): |
| | 7831-7846 |
| V-ATPase | WO 01/14572 |
| Super promoter | WO 95/14098 |
| G-box proteins | WO 94/12015 |

For the identification of functionally equivalent promoters, the promoter strength and/or expression pattern of a candidate promoter may be analysed for example by operably linking the promoter to a reporter gene and assay the expression level and pattern of the reporter gene in various tissues of the plant. Suitable well-known reporter genes include for example beta-glucuronidase or beta galactosidase. The promoter activity is assayed by measuring the enzymatic activity of the beta-glucuronidase or beta-galactosidase. The promoter strength and/or expression pattern may then be compared to that of a reference promoter (such as the one used in the methods of the present invention). Alternatively, promoter strength may be assayed by quantifying mRNA levels or by comparing mRNA levels of the nucleic acid used in the methods of the present invention, with mRNA levels of housekeeping genes such as 18S rRNA, using methods known in the art, such as Northern blotting with densitometric analysis of autoradiograms, quantitative real-time PCR or RT-PCR (Heid et al., 1996 Genome Methods 6: 986-994). Generally by “weak promoter” is intended a promoter that drives expression of a coding sequence at a low level. By “low level” is intended at levels of about 1/10,000 transcripts to about 1/100,000 transcripts, to about 1/500,0000 transcripts per cell. Conversely, a “strong promoter” drives expression of a coding sequence at high level, or at about 1/10 transcripts to about 1/100 transcripts to about 1/1,000 transcripts per cell.

Optionally, one or more terminator sequences may also be used in the construct introduced into a plant. The term “terminator” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene. Additional regulatory elements may include transcriptional as well as translational enhancers. Those skilled in the art will be aware of terminator and enhancer sequences that may be suitable for use in performing the invention. Such sequences would be known or may readily be obtained by a person skilled in the art.

Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. As used herein, the term “selectable marker”, “selectable marker gene” or “reporter gene” includes any gene that confers a phenotype on a cell in which it is expressed to facilitate the identification and/or selection of cells that are transfected or transformed with a nucleic acid construct of the invention. These marker genes enable the identification of a successful transfer of the nucleic acid molecules via a series of different principles. Suitable markers may be selected from markers that confer antibiotic or herbicide resistance, that introduce a new metabolic trait or that allow visual selection. Examples of selectable marker genes include genes conferring resistance to antibiotics (such as nptII that phosphorylates neomycin and kanamycin, or hpt, phosphorylating hygromycin, or genes conferring resistance to, for example, bleomycin, streptomycin, tetracyclin, chloramphenicol, ampicillin, gentamycin, geneticin (G418), spectinomycin or blasticidin), to herbicides (for example bar which provides resistance to Basta; aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xlose isomerase for the utilisation of xylose, or antinutritive markers such as the resistance to 2-deoxyglucose). Expression of visual marker genes results in the formation of colour (for example β-glucuronidase, GUS or β-galactosidase with its coloured substrates, for example X-Gal), luminescence (such as the luciferin/luciferase system) or fluorescence (Green Fluorescent Protein, GFP, and derivatives thereof). This list represents only a small number of possible markers. The skilled worker is familiar with such markers. Different markers are preferred, depending on the organism and the selection method.

It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid can be identified for example by selection (for example, cells which have integrated the selectable marker survive whereas the other cells die).

Since the marker genes, particularly genes for resistance to antibiotics and herbicides, are no longer required or are undesired in the transgenic host cell once the nucleic acids have been introduced successfully, the process according to the invention for introducing the nucleic acids advantageously employs techniques which enable the removal or excision of these marker genes. One such a method is what is known as co-transformation. The co-transformation method employs two vectors simultaneously for the transformation, one vector bearing the nucleic acid according to the invention and a second bearing the marker gene(s). A large proportion of transformants receives or, in the case of plants, comprises (up to 40% or more of the transformants), both vectors. In case of transformation with Agrobacteria, the transformants usually receive only a part of the vector, i.e. the sequence flanked by the T-DNA, which usually represents the expression cassette. The marker genes can subsequently be removed from the transformed plant by performing crosses. In another method, marker genes integrated into a transposon are used for the transformation together with desired nucleic acid (known as the Ac/Ds technology). The transformants can be crossed with a transposase source or the transformants are transformed with a nucleic acid construct conferring expression of a transposase, transiently or stable. In some cases (approx. 10%), the transposon jumps out of the genome of the host cell once transformation has taken place successfully and is lost. In a further number of cases, the transposon jumps to a different location. In these cases the marker gene must be eliminated by performing crosses. In microbiology, techniques were developed which make possible, or facilitate, the detection of such events. A further advantageous method relies on what is known as recombination systems; whose advantage is that elimination by crossing can be dispensed with. The best-known system of this type is what is known as the Cre/lox system. Cre1 is a recombinase that removes the sequences located between the loxP sequences. If the marker gene is integrated between the loxP sequences, it is removed once transformation has taken place successfully, by expression of the recombinase. Further recombination systems are the HIN/HIX, FLP/FRT and REP/STB system (Tribble et al., J. Biol. Chem., 275, 2000: 22255-22267; Velmurugan et al., J. Cell Biol., 149, 2000: 553-566). A site-specific integration into the plant genome of the nucleic acid sequences according to the invention is possible. Naturally, these methods can also be applied to microorganisms such as yeast, fungi or bacteria.

The present invention also encompasses plants or parts thereof (including seeds) obtainable by the methods according to the present invention. The present invention therefore provides plants obtainable by the method according to the present invention, which plants or plant parts have introduced therein a DEL1f nucleic acid or variant thereof.

The invention also provides a method for the production of transgenic plants having increased yield and/or increased stress resistance, comprising introduction and expression in a plant of a DEL1f nucleic acid or a variant thereof.

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either
a) the nucleic acid sequences encoding proteins useful in the methods of the invention, or
b) genetic control sequence(s) which is operably linked with the nucleic acid sequence according to the invention, for example a promoter, or
c) a) and b)
   are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815.

More specifically, the present invention provides a method for the production of transgenic plants having increased yield and/or increased stress resistance, which method comprises:
(i) introducing and expressing in a plant or plant cell a DEL1f nucleic acid or variant thereof; and
(ii) cultivating the plant cell under conditions promoting plant growth and development.

The nucleic acid may be introduced directly into a plant cell or into the plant itself (including introduction into a tissue, organ or any other part of a plant). According to a preferred feature of the present invention, the nucleic acid is preferably introduced into a plant by transformation.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al. (1985) Bio/Technol 3, 1099-1102); microinjection into plant material (Crossway A et al., (1986) Mol. Gen. Genet. 202: 179-185); DNA or RNA-coated particle bombardment (Klein T M et al., (1987) Nature 327: 70) infection with (non-integrative) viruses and the like. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least on the flower primordial. The plant is subsequently grown on until the seeds of the treated plant are obtained (Clough and Bent, Plant J. (1998) 16, 735-743). Methods for Agrobacterium-mediated transformation of rice include well known methods for rice transformation, such as those described in any of the following: European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for transforming Agrobacterium tumefaciens, for example pBinl9 (Bevan et al., Nucl. Acids Res. 12 (1984) 8711). Agrobacteria transformed by such a vector can then be used in known manner for the transformation of plants, such as plants used as a model, like Arabidopsis (Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Hofgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic [Feldman, K A and Marks M D (1987). Mol Gen

Genet. 208:274-289; Feldmann K (1992). In: C Koncz, N-H Chua and J Shell, eds, Methods in Arabidopsis Research. Word Scientific, Singapore, pp. 274-289]. Alternative methods are based on the repeated removal of the inflorescences and incubation of the excision site in the center of the rosette with transformed agrobacteria, whereby transformed seeds can likewise be obtained at a later point in time (Chang (1994). Plant J. 5: 551-558; Katavic (1994). Mol Gen Genet, 245: 363-370). However, an especially effective method is the vacuum infiltration method with its modifications such as the “floral dip” method. In the case of vacuum infiltration of Arabidopsis, intact plants under reduced pressure are treated with an agrobacterial suspension [Bechthold, N (1993). C R Acad Sci Paris Life Sci, 316: 1194-1199], while in the case of the “floral dip” method the developing floral tissue is incubated briefly with a surfactant-treated agrobacterial suspension [Clough, S J und Bent, A F (1998). The Plant J. 16, 735-743]. A certain proportion of transgenic seeds are harvested in both cases, and these seeds can be distinguished from non-transgenic seeds by growing under the above-described selective conditions. In addition the stable transformation of plastids is of advantages because plastids are inherited maternally is most crops reducing or eliminating the risk of transgene flow through pollen. The transformation of the chloroplast genome is generally achieved by a process which has been schematically displayed in Klaus et al., 2004 [Nature Biotechnology 22 (2), 225-229]. Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview is given in Bock (2001) Transgenic plastids in basic research and plant biotechnology. J Mol. Biol. 2001 Sep. 21; 312 (3):425-38 or Maliga, P (2003) Progress towards commercialization of plastid transformation technology. Trends Biotechnol. 21, 20-28. Further biotechnological progress has recently been reported in form of marker free plastid transformants, which can be produced by a transient co-integrated maker gene (Klaus et al., 2004, Nature Biotechnology 22(2), 225-229).

The genetically modified plant cells can be regenerated via all methods with which the skilled worker is familiar. Suitable methods can be found in the abovementioned publications by S. D. Kung and R. Wu, Potrykus or Hofgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques.

The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid encoding a DELL protein as defined hereinabove. Preferred host cells according to the invention are plant cells.

Host plants for the nucleic acids or the vector used in the method according to the invention, the expression cassette or construct or vector are, in principle, advantageously all plants, which are capable of synthesizing the polypeptides used in the inventive method.

A transgenic plant for the purposes of the invention is thus understood as meaning, as above, that the nucleic acids used in the method of the invention are not at their natural locus in the genome of said plant, it being possible for the nucleic acids to be expressed homologously or heterologously. However, as mentioned, transgenic also means that, while the nucleic acids according to the invention or used in the inventive method are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. Preferred transgenic plants are mentioned herein.

The invention also extends to harvestable parts of a plant such as, but not limited to seeds, leaves, fruits, flowers, stems, rhizomes, tubers and bulbs. The invention furthermore relates to products derived, preferably directly derived, from a harvestable part of such a plant, such as dry pellets or powders, oil, fat and fatty acids, starch or proteins.

The present invention also encompasses use of plant mutants with decreased expression of a DEL1 gene for improving the growth characteristics of plants, in particular in increasing yield and/or increasing stress resistance.

The present invention furthermore encompasses use of DEL1 nucleic acids or variants thereof and use of DEL1 polypeptides or homologues thereof.

One such use relates to improving the growth characteristics of plants, in particular in increasing yield and/or increasing stress resistance. The yield increase comprises at least one of increased biomass, increased total weight of seeds and increased number of filled seeds.

DEL1 nucleic acids or variants thereof, or DEL1 polypeptides or homologues thereof may find use in breeding programmes in which a DNA marker is identified which may be genetically linked to a DEL1 gene or variant thereof. The DEL1 nucleic acids/genes or variants thereof, or DEL1 polypeptides or homologues thereof may be used to define a molecular marker. This DNA or protein marker may then be used in breeding programmes to select plants having increased yield and/or increased stress resistance. The DEL1 gene or variant thereof may, for example, be a nucleic acid as represented by any one of the sequences listed in Table A of Example 1.

Allelic variants of a DEL1 nucleic acid/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield and/or increased stress resistance. Selection is typically carried out by monitoring growth performance of plants containing different allelic variants of the sequence in question, for example, different allelic variants of any one of the sequences listed in Table A of Example 1. Growth performance may be monitored in a greenhouse or in the field. Further optional steps include crossing plants, in which the superior allelic variant was identified, with another plant. This could be used, for example, to make a combination of interesting phenotypic features.

A DEL1 nucleic acid or variant thereof may also be used as probes for genetically and physically mapping the genes that they are a part of, and as markers for traits linked to those genes. Such information may be useful in plant breeding in order to develop lines with desired phenotypes. Such use of DEL1 nucleic acids or variants thereof requires only a nucleic acid sequence of at least 15 nucleotides in length. The DEL1 nucleic acids or variants thereof may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the DEL1 nucleic acids or variants thereof. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the DEL1 nucleic acid or variant thereof in the genetic map previously obtained using this population (Botstein et al. (1980) Am. J. Hum. Genet. 32: 314-331).

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridization (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med. 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having increased yield and/or increased stress resistance, as described hereinbefore. These advantageous growth characteristics may also be combined with other economically advantageous traits, such as further yield-enhancing traits, further tolerance to various stresses, traits modifying various architectural features and/or biochemical and/or physiological features.

### DESCRIPTION OF FIGURES

The present invention will now be described with reference to the following figures in which:

FIG. 1 shows the typical domain structure of DEL1 polypeptides. DEL1 proteins comprise 2 E2F_TDP domains (Pfam PF02319, Interpro IPRO03316), which represent a winged-helix DNA-binding domain. The domains are indicated in bold.

FIG. 2 shows a multiple alignment of various DEL1 proteins. The asterisks represent identical amino acids in the various sequences, the colons represent highly conserved substitutions, and the dots represent less conserved substitutions.

FIG. 3 shows a binary vector p037, for expression in Oryza sativa of an Arabidopsis thaliana DEL1f coding sequence under the control of a seed-specific promoter (internal reference PRO0218).

FIG. 4 details examples of sequences useful in performing the methods according to the present invention.

FIG. 5 represents biomass per plant measurements in response to increasing UV-B doses. 18-day-old plants were irradiated with indicated doses of UV-B. Biomass of plants were measured 8 days after UV-B treatments. Values indicate means±SE. DEL1.2 and DEL1.4 correspond to the DEL1 overepressing lines #2 and #4, respectively. DEL1 KO corresponds to the del1-1 knock-out line.

FIG. 6 shows cyclobutylpyrimidine dimer (CPD) photoproducts measurements. The total level of CPDs were measured for the 5^{th} leaf of 18-day-old plants either directly after 5 hours of UV-B treatment (A), or after an additional exposure to white light, enabling DNA photorepair (B). CPD levels are plotted relatively to the level measured in the Col-0 plants.

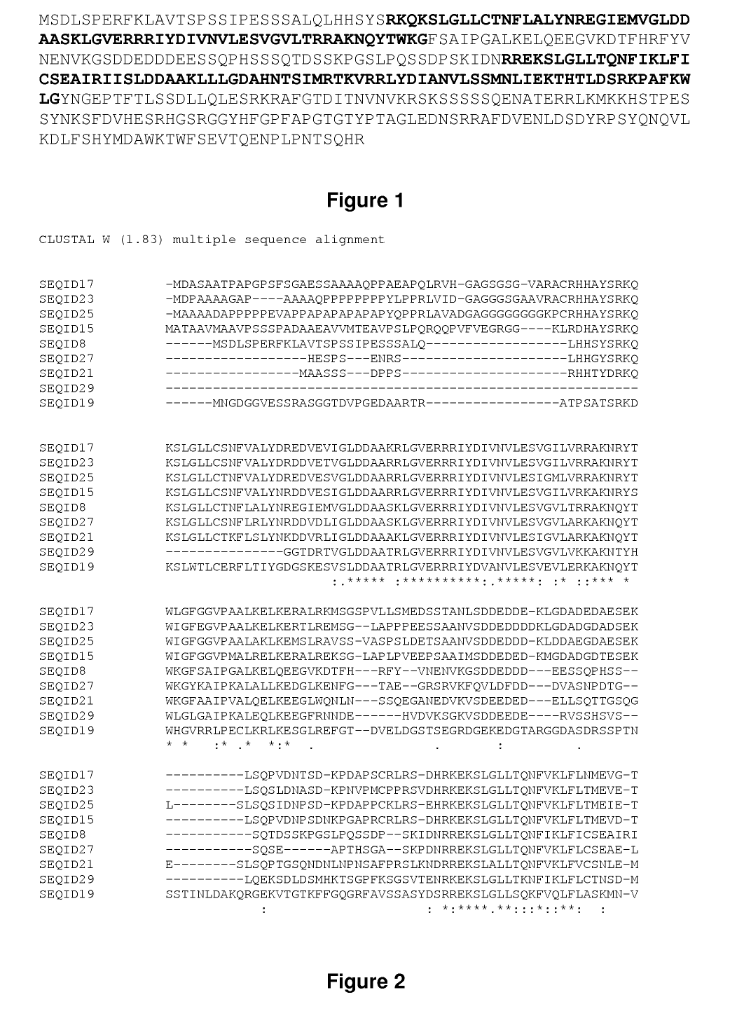

### EXAMPLES

The present invention will now be described with reference to the following examples, which are by way of illustration alone. The following examples are not intended to completely define or otherwise limit the scope of the invention.

### Example 1

### Identification of sequences Related to SEQ ID NO: 7 and SEQ ID NO: 8

Sequences (full length cDNA, ESTs or genomic) related to SEQ ID NO: 7 and/or protein sequences related to SEQ ID NO: 8 were identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. The polypeptide encoded by SEQ ID NO: 8 was used for the TBLASTN algorithm, with default settings and the filter to ignore low complexity sequences set off. The output of the analysis was viewed by pairwise comparison, and ranked according to the probability score (E-value), where the score reflects the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search.

In addition to the publicly available nucleic acid sequences available at NCBI, proprietary sequence databases are also searched following the same procedure as described herein above.

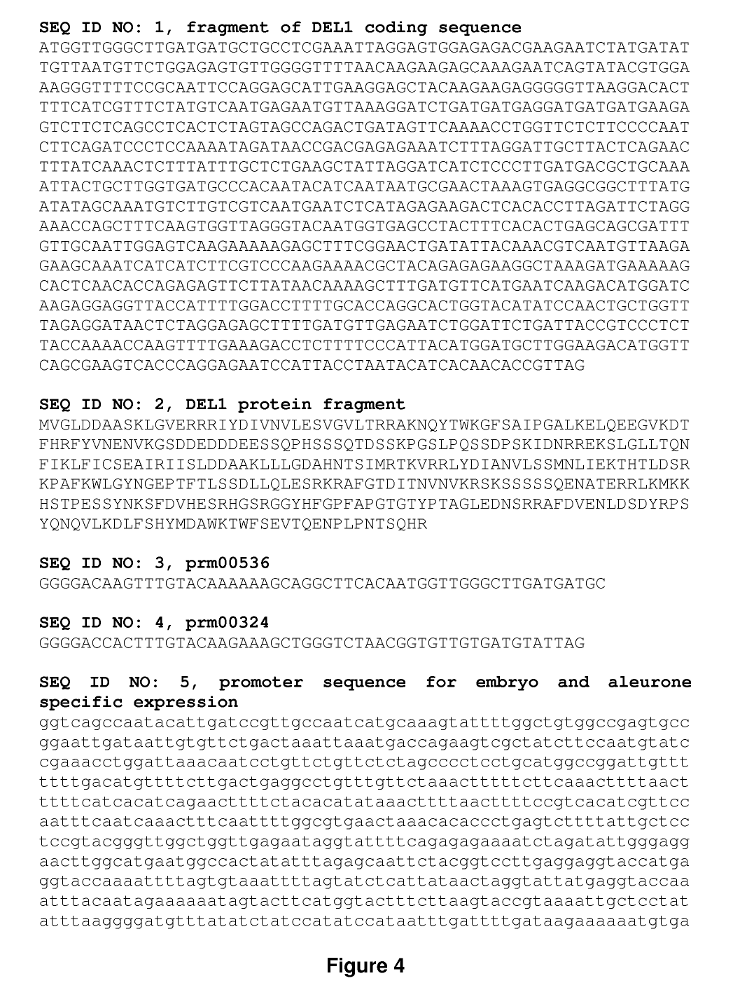

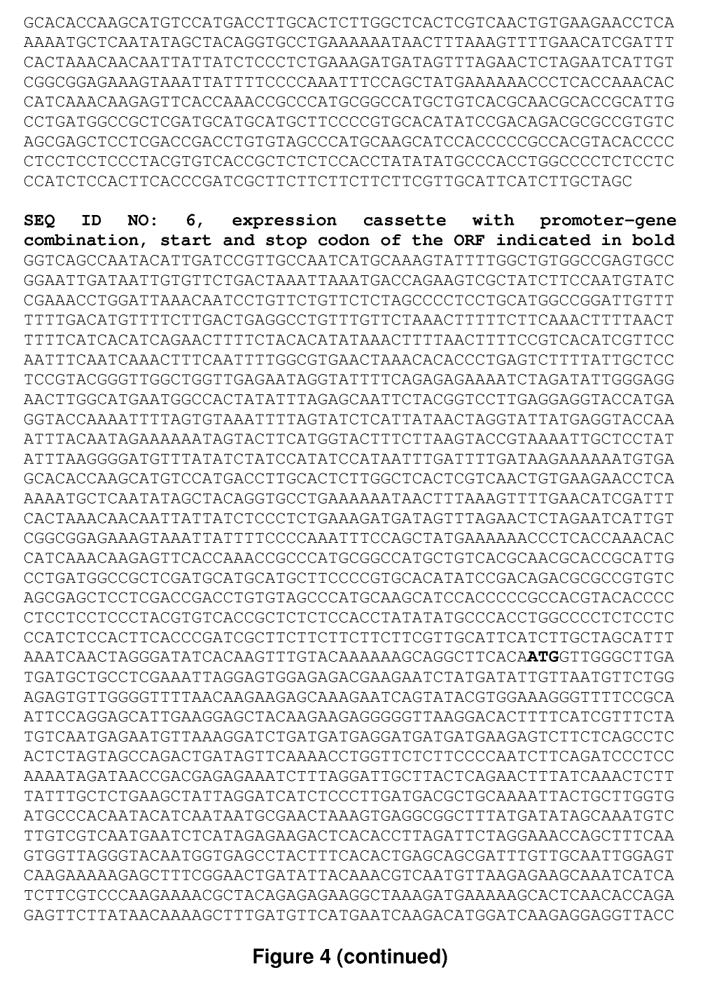

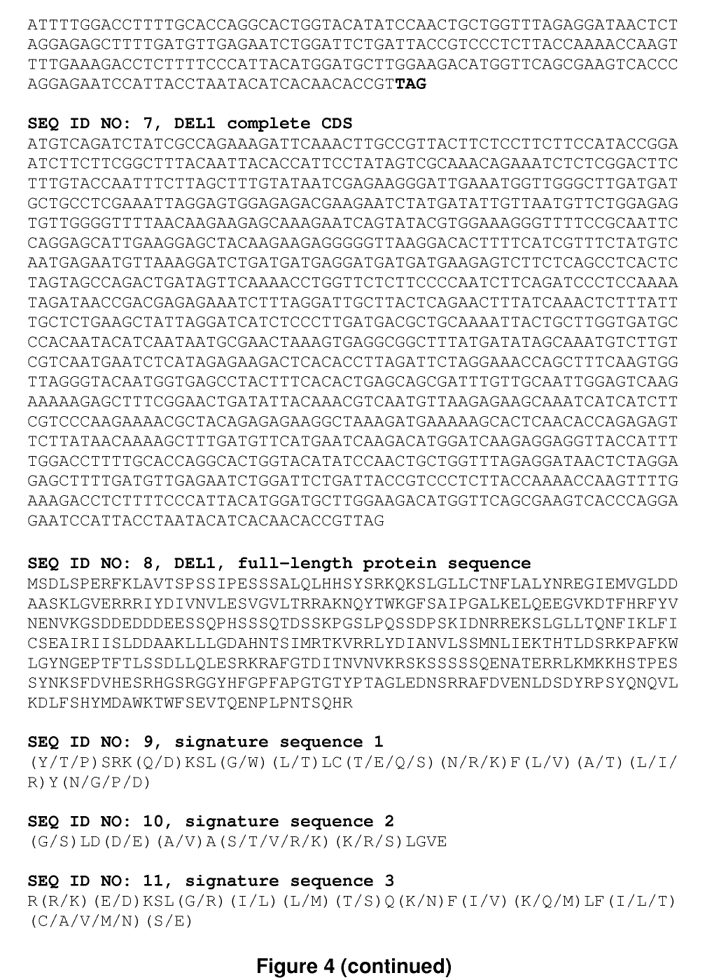

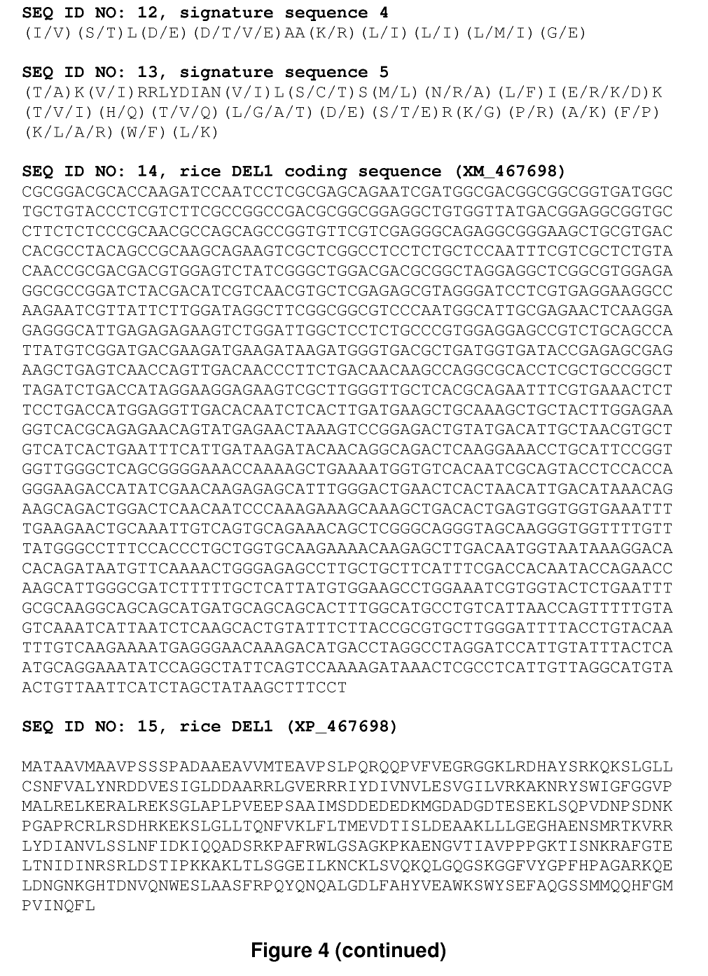

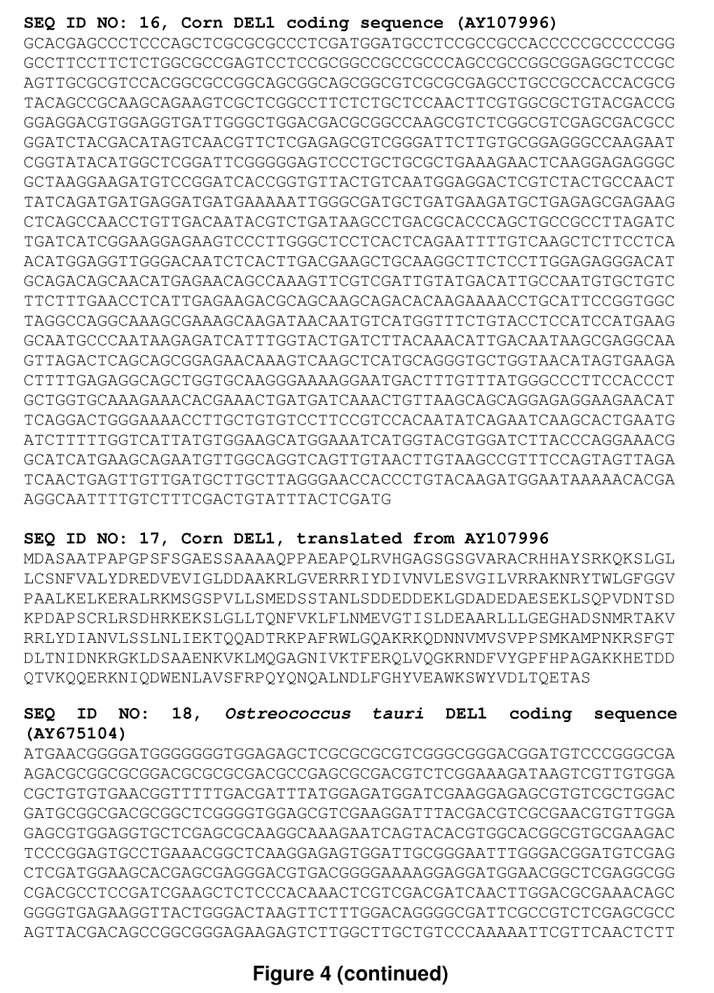

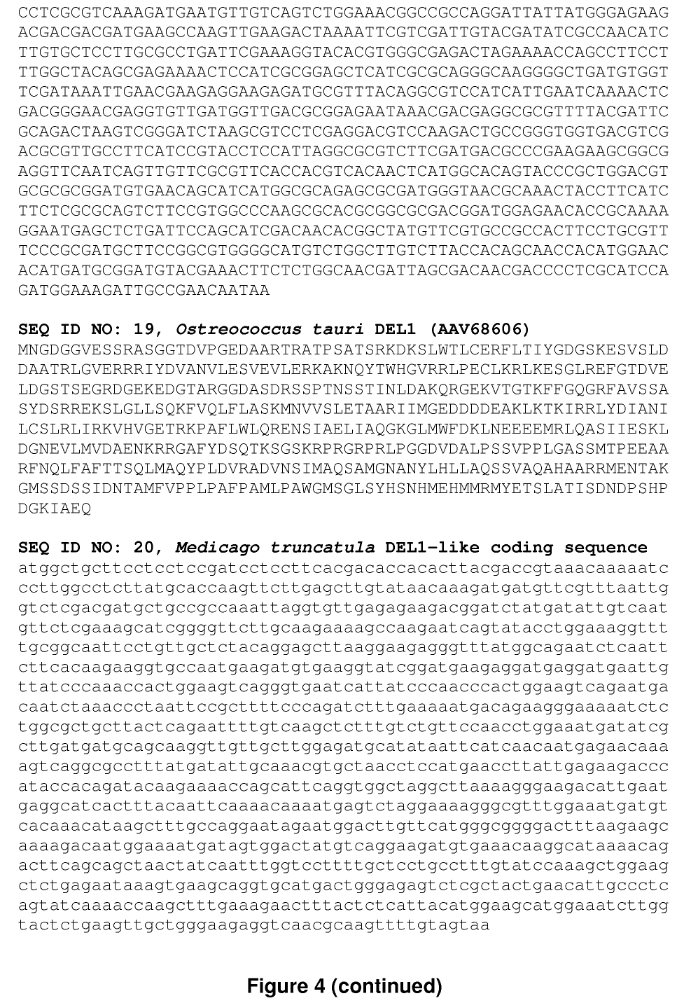

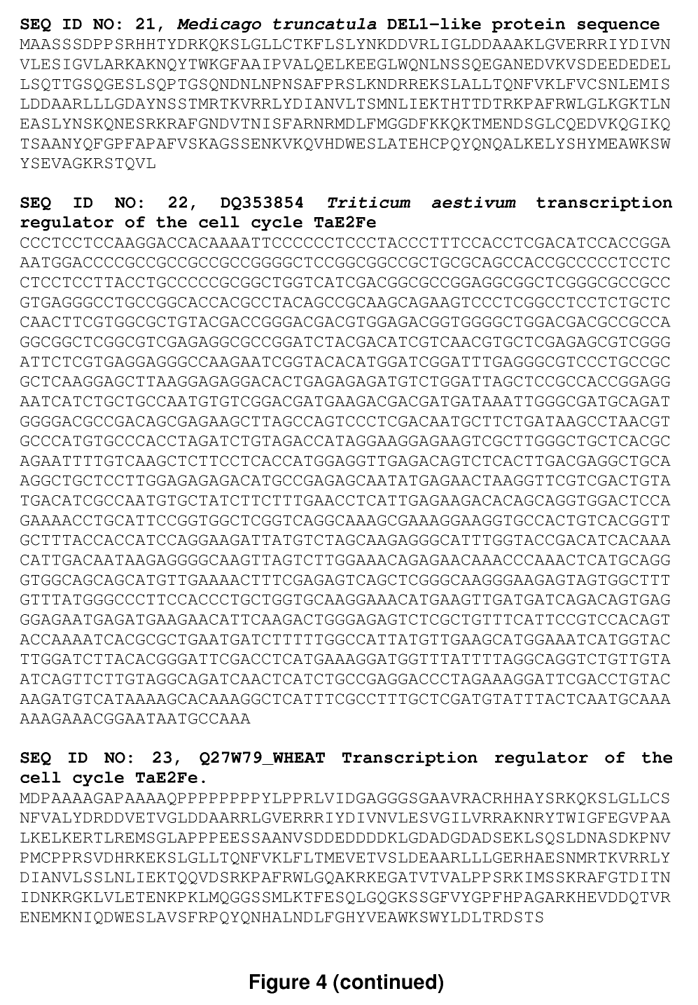

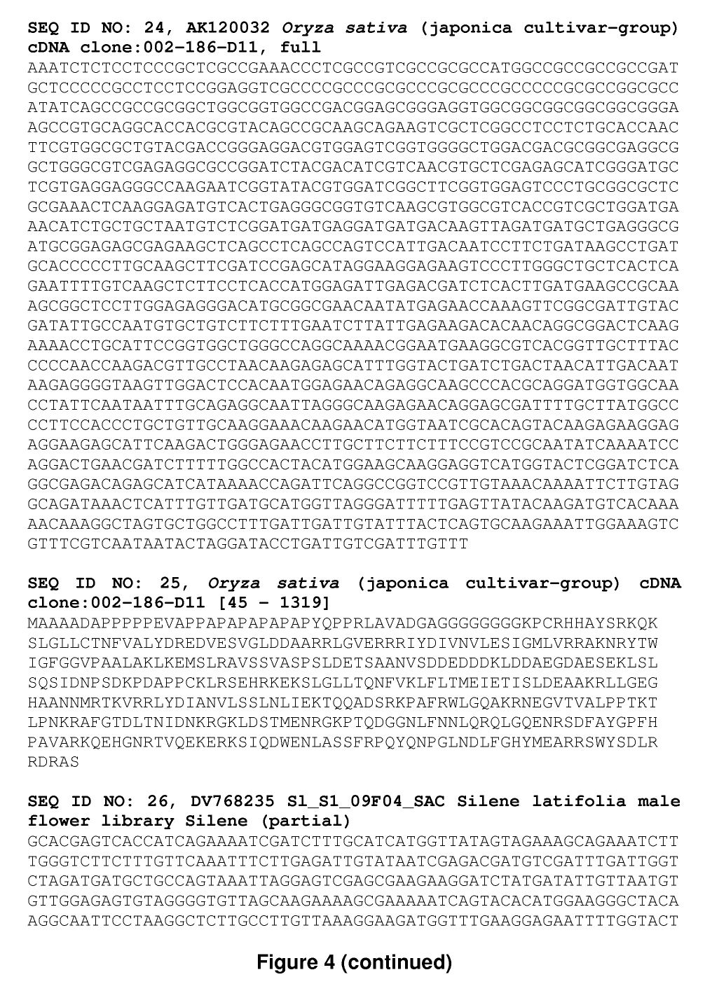

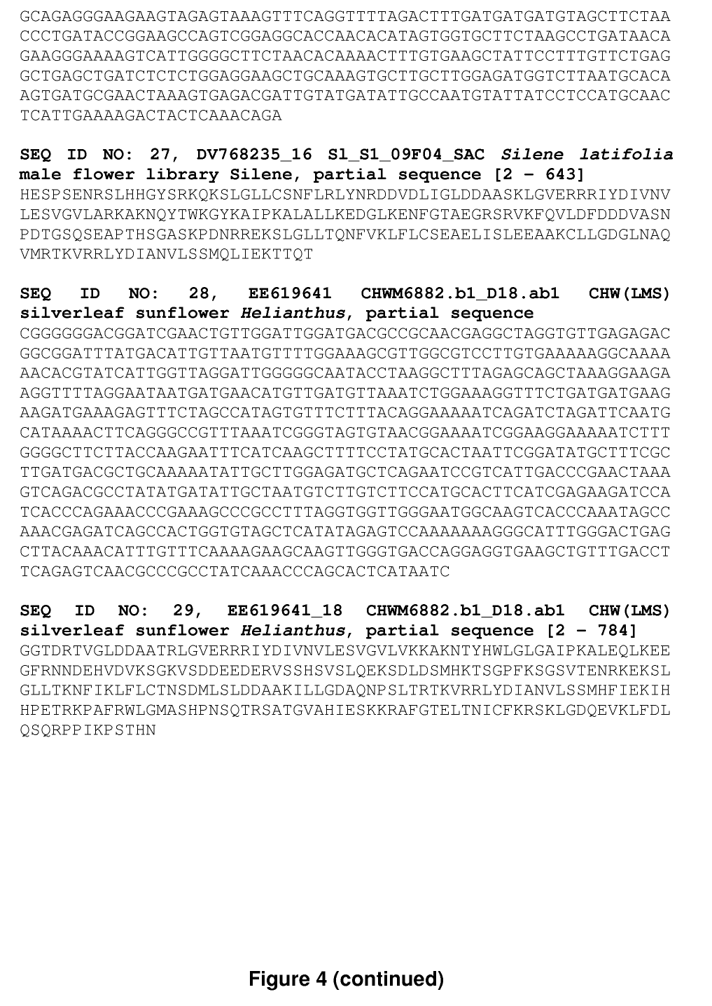

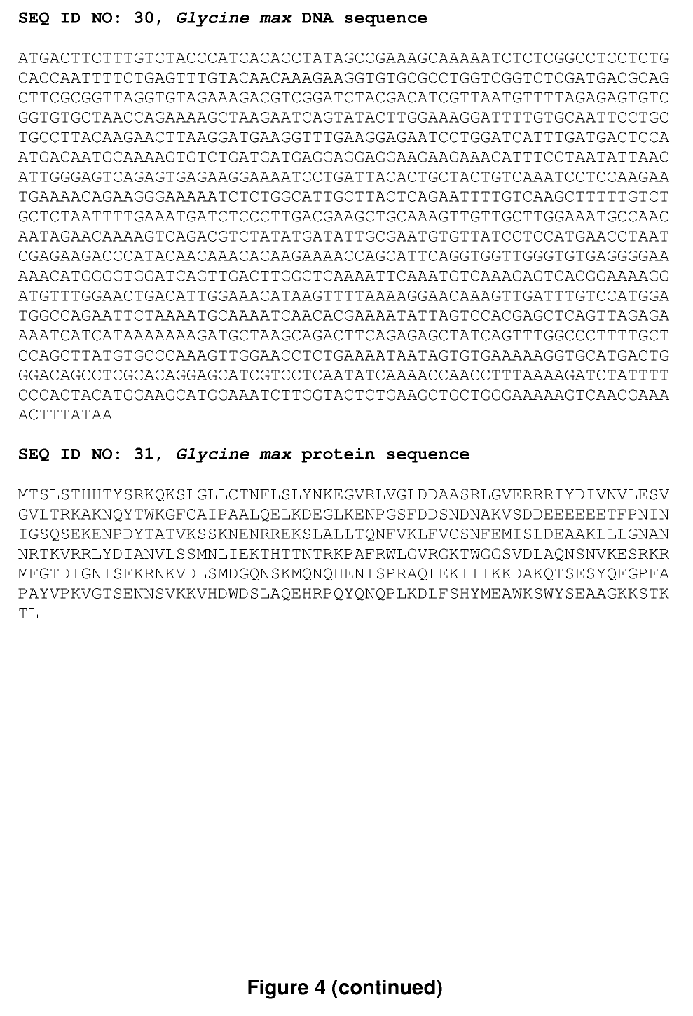

Table A provides a list of nucleic acid and protein sequences related to the nucleic acid sequence as represented by SEQ ID NO: 7 and the protein sequence represented by SEQ ID NO: 8, see also FIG. 4.

TABLE A
Nucleic acid sequences related to the nucleic acid sequence (SEQ ID NO: 7)
useful in the methods of the present invention, and the corresponding
deduced polypeptides.

| | | | | Database | |
|---|---|---|---|---|---|
| | Source | Nucleic acid | Poly-peptide | accession | |
| Name | organism | SEQ ID NO: | SEQ ID NO: | number | Status |
| DEL1 | Arabidopsis | 7 | 8 | NM_114685 | Full length |
| | thaliana | | | | |
| OsDEL1 | Oryza sativa | 14 | 15 | XM_467698 | Full length |
| ZmDEL1 | Zea mays | 16 | 17 | AY107996 | Full length |
| OtDEL1 | Ostreococcus | 18 | 19 | AY675104 | Full length |
| | tauri | | | | |
| MtDEL1-like | Medicago | 20 | 21 | / | Full length |
| | truncatula | | | | |
| TaE2Fe | Triticum | 22 | 23 | DQ353854 | Full length |
| | aestivum | | | | |
| OsDEL1-like | Oryza sativa | 24 | 25 | AK120032 | Full length |
| SIDEL1-like | Silene latifolia | 26 | 27 | DV768235 | partial |
| HDEL1-like | Helianthus sp. | 28 | 29 | EE619641 | partial |
| GmDEL1-like | Glycine max | 30 | 31 | Gm59592851 | Full length |

### Example 2

### Alignment of Relevant Polypeptide Sequences

AlignX from the Vector NTI (Invitrogen) is based on the popular Clustal algorithm of progressive alignment (Thompson et al. (1997) Nucleic Acids Res 25:4876-4882; Chema et al. (2003). Nucleic Acids Res 31:3497-3500). Default values are for the gap open penalty of 10, for the gap extension penalty of 0.1 and the selected weight matrix is Blosum 62 (if polypeptides are aligned).

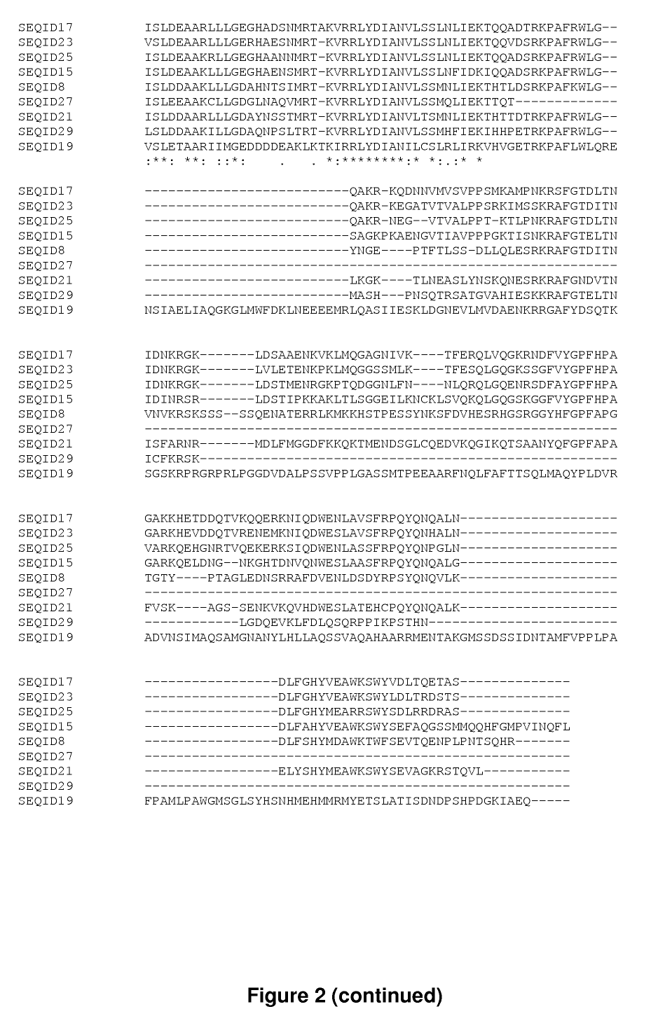

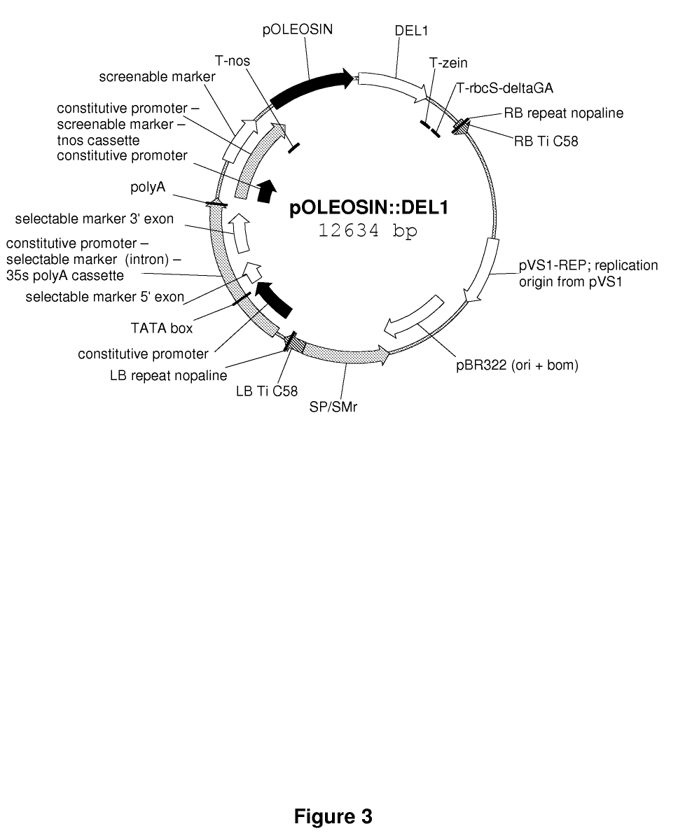

The result of a multiple sequence alignment using polypeptides relevant in identifying the ones useful in performing the methods of the invention is shown in FIG. 2. The two conserved E2F_TDP domains can be easily identified.

### Example 3

### Calculation of Global Percentage Identity between Polypeptide Sequences Useful in Performing the Methods of the Invention

Global percentages of similarity and identity between full length polypeptide sequences useful in performing the methods of the invention were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:
Scoring matrix: Blosum62
First Gap: 12
Extending gap: 2

Results of the software analysis are shown in Table B for the global similarity and identity over the full length of the polypeptide sequences (excluding the partial polypeptide sequences). Percentage identity is given above the diagonal and percentage similarity is given below the diagonal.

The percentage identity between the polypeptide sequences useful in performing the methods of the invention can be as low as 27.5% amino acid identity compared to SEQ ID NO: 8.

TABLE B
MatGAT results for global similarity and identity over
the full length of the polypeptide sequences.

| | 1 | 2 | 3 | 4 | 5 | 6 | 7 | 8 | 9 |
|---|---|---|---|---|---|---|---|---|---|
| 1. SEQID8 | | 42.9 | 44.2 | 27.5 | 51.5 | 45.6 | 43.4 | 36.2 | 35.7 |
| 2. SEQID15 | 60.8 | | 63.6 | 28.6 | 45.4 | 63.7 | 60.2 | 29.6 | 31.4 |
| 3. SEQID17 | 63.1 | 76.4 | | 29.5 | 45.1 | 74.8 | 69.4 | 30.7 | 31.8 |
| 4. SEQID19 | 46.5 | 44.5 | 45.1 | | 30.6 | 28.8 | 28.4 | 21.9 | 24.0 |
| 5. SEQID21 | 69.7 | 60.8 | 61.5 | 44.1 | | 46.8 | 43.1 | 36.4 | 37.3 |
| 6. SEQID23 | 63.0 | 75.1 | 85.2 | 42.7 | 62.8 | | 71.8 | 31.0 | 32.3 |
| 7. SEQID25 | 61.9 | 75.3 | 82.6 | 43.1 | 60.5 | 82.4 | | 30.4 | 32.9 |
| 8. SEQID27 | 42.7 | 35.6 | 38.0 | 29.4 | 43.4 | 38.2 | 37.2 | | 35.6 |
| 9. SEQID29 | 47.6 | 41.7 | 43.2 | 35.6 | 49.6 | 42.4 | 43.1 | 53.3 | |

### Example 4

### Identification of Domains Comprised in Polypeptide Sequences Useful in Performing the Methods of the Invention

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, ProDom and Pfam, Smart and TIGRFAMs. Interpro is hosted at the European Bioinformatics Institute in the United Kingdom.

The results of the InterPro scan of the polypeptide sequence as represented by SEQ ID NO: 8 are presented in Table C.

TABLE C
InterPro scan results of the polypeptide
sequence as represented by SEQ ID NO: 8

| Database | Accession number | Accession name |
|---|---|---|
| InterPro | IPR003316 | E2F_TDP |

### Example 5

### Topology Prediction of the Polypeptide Sequences Useful in Performing the Methods of the Invention (Subcellular Localization, Transmembrane . . . )

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

For the sequences predicted to contain an N-terminal presequence a potential cleavage site can also be predicted.

A number of parameters were selected, such as organism group (non-plant or plant), cutoff sets (none, predefined set of cutoffs, or user-specified set of cutoffs), and the calculation of prediction of cleavage sites (yes or no).

The results of TargetP 1.1 analysis of the polypeptide sequence as represented by SEQ ID NO: 8 are presented Table D. The “plant” organism group has been selected, no cutoffs defined, and the predicted length of the transit peptide requested. There is no clear prediction of the subcellular localisation, only a weak prediction for a chloroplastic localisation (reliability class 3, which is fairly low). DEL1 proteins therefore may be located in the cytoplasm or the nucleus.

TABLE D
TargetP 1.1 analysis of the polypeptide
sequence as represented by SEQ ID NO: 8

| | |
|---|---|
| Length (AA) | 403 |
| Chloroplastic transit peptide | 0.269 |
| Mitochondrial transit peptide | 0.063 |
| Secretory pathway signal peptide | 0.053 |
| Other subcellular targeting | 0.721 |
| Predicted Location | / |
| Reliability class | 3 |
| Predicted transit peptide length | 12 |

Many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
PSORT (and in particular WOLF PSORT) for subcellular localisation, hosted by the Brinkman Laboratory at Simon Fraser University

When analysed with PSORT, using the default values, the DEL1 protein of SEQ ID NO: 8 is predicted to be localised in the nucleus (output data: nucl: 10, chlo: 1, cyto: 1, pero: 1, cysk: 1).

### Example 6

### DEL1 Activity Assay

DEL1 activity may be assayed for example by determining DNA binding activity, several assays are available and well known in the art (for example the Electric Mobility Shift Assay (EMSA) described in Kosugi & Ohashi (J. Biol. Chem. 19, 16553-16558, 2002a) or in Kosugi & Ohashi (Plant J. 29, 45-59, 2002b). Another aspect of DEL1 activity, namely repression of E2F-regulated promoters, may be assayed by measuring transcription of reporter genes under control of E2F-regulated promoters in the presence or absence of DEL1 proteins (for example as described by Kosugi and Ohshi (2002a).

Furthermore, expression of a nucleic acid encoding the polypeptide of SEQ ID NO: 2, representing a fragment of a DEL1 polypeptide, in plants, and in particular in rice, has the effect of increasing yield of the transgenic plant when compared to control plants, wherein increased yield comprises at least total weight of seeds or number of filled seeds. In addition, inhibiting DEL1 expression in transgenic plants has the effect of increasing stress resistance of plants, measured as increased biomass compared to control plants upon UV-B radiation treatment (see Examples below).

### Example 7

### Gene Cloning of DEL1f

The Arabidopsis thaliana DEL1 gene fragment (DEL1) was amplified by PCR using as template an Arabidopsis thaliana seedling cDNA library (Invitrogen, Paisley, UK). After reverse transcription of RNA extracted from seedlings, the cDNAs were cloned into pCMV Sport 6.0. Average insert size of the bank was 1.5 kb and the original number of clones was of the order of 1.59×10⁷ cfu. Original titer was determined to be 9.6×10⁵ cfu/ml after first amplification of 6×10¹¹ cfu/ml. After plasmid extraction, 200 ng of template was used in a 50 μl PCR mix. Primers prm00536 (SEQ ID NO: 3; sense, start codon in bold, AttB1 site in italic: 5′-ggggacaagtttgtacaaaaaagcaggcttcacaatggttgggcttgatgatgc 3′) and prm00324 (SEQ ID NO: 4; reverse, complementary, stop codon in bold, AttB2 site in italic: 5′ ggggaccactttgtacaagaaagctgggtctaacggtgttgtgatgtattag 3′), which include the AttB sites for Gateway recombination, were used for PCR amplification. PCR was performed using Hifi Taq DNA polymerase in standard conditions. A PCR fragment of 1107 bp (including attB sites) was amplified and purified also using standard methods. The first step of the Gateway procedure, the BP reaction, was then performed, during which the PCR fragment recombines in vivo with the pDONR201 plasmid to produce, according to the Gateway terminology, an “entry clone”, p042. Plasmid pDONR201 was purchased from Invitrogen, as part of the Gateway® technology.

### Example 8

### Vector Construction

The entry clone p042 was subsequently used in an LR reaction with p00831, a destination vector used for Oryza sativa transformation. This vector contains as functional elements within the T-DNA borders: a plant selectable marker; a screenable marker expression cassette; and a Gateway cassette intended for LR in vivo recombination with the sequence of interest already cloned in the entry clone. A promoter for embryo and aleurone specific expression (PRO0218, SEQ ID NO: 5) was located upstream of this Gateway cassette. The expression cassette PRO0218::DEL1-fragment is given in SEQ ID NO: 6

After the LR recombination step, the resulting expression vector, p037 for DEL1f (FIG. 3) was transformed into Agrobacterium strain LBA4044 and subsequently to Oryza sativa plants. Transformed rice plants were allowed to grow and were then examined for the parameters described in Example 3.

### Example 9

### Evaluation and Results of DEL1f Under the Control of the PRO0218 Promoter

Approximately 15 to 20 independent T0 rice transformants were generated. The primary transformants were transferred from a tissue culture chamber to a greenhouse for growing and harvest of T1 seed. Four events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The selected T1 plants were transferred to a greenhouse. Each plant received a unique barcode label to link unambiguously the phenotyping data to the corresponding plant. The selected T1 plants were grown on soil in 10 cm diameter pots under the following environmental settings: photoperiod=11.5 h, daylight intensity=30,000 lux or more, daytime temperature=28° C. or higher, night time temperature=22° C., relative humidity=60-70%. Care was taken that the plants were grown under optimal conditions. Transgenic plants and the corresponding nullizygotes were grown side-by-side at random positions. From the stage of sowing until the stage of maturity the plants were passed several times through a digital imaging cabinet. At each time point digital images (2048×1536 pixels, 16 million colours) were taken of each plant from at least 6 different angles.

The mature primary panicles were harvested, bagged, barcode-labelled and then dried for three days in the oven at 37° C. The panicles were then threshed and all the seeds collected. The filled husks were separated from the empty ones using an air-blowing device. After separation, both seed lots were then counted using a commercially available counting machine. The empty husks were discarded. The filled husks were weighed on an analytical balance and the cross-sectional area of the seeds was measured using digital imaging. This procedure resulted in the set of the following seed-related parameters:

The number of filled seeds was determined by counting the number of filled husks that remained after the separation step. The total seed weight was measured by weighing all filled husks harvested from a plant. Total seed number per plant was measured by counting the number of husks harvested from a plant. Thousand Kernel Weight (TKW) is extrapolated from the number of filled seeds counted and their total weight. Harvest index is defined as the ratio between the total seed weight and the above-ground area (mm²), multiplied by a factor 10⁶. These parameters were derived in an automated way from the digital images using image analysis software and were analysed statistically. Individual seed parameters (including width, length, area, weight) were measured using a custom-made device consisting of two main components, a weighing and imaging device, coupled to software for image analysis.

A two factor ANOVA (analyses of variance) corrected for the unbalanced design was used as statistical model for the overall evaluation of plant phenotypic characteristics. An F-test was carried out on all the parameters measured of all the plants of all the events transformed with that gene. The F-test was carried out to check for an effect of the gene over all the transformation events and to verify for an overall effect of the gene, also named herein “global gene effect”. If the value of the F test shows that the data are significant, than it is concluded that there is a “gene” effect, meaning that not only presence or the position of the gene is causing the effect. The threshold for significance for a true global gene effect is set at 5% probability level for the F test.

To check for an effect of the genes within an event, i.e., for a line-specific effect, a t-test was performed within each event using data sets from the transgenic plants and the corresponding null plants. “Null plants” or “null segregants” or “nullizygotes” are the plants treated in the same way as the transgenic plant, but from which the transgene has segregated. Null plants can also be described as the homozygous negative transformed plants. The threshold for significance for the t-test is set at 10% probability level. The results for some events can be above or below this threshold. This is based on the hypothesis that a gene might only have an effect in certain positions in the genome, and that the occurrence of this position-dependent effect is not uncommon. This kind of gene effect is also named herein a “line effect of the gene”. The p-value is obtained by comparing the t-value to the t-distribution or alternatively, by comparing the F-value to the F-distribution. The p-value then gives the probability that the null hypothesis (i.e., that there is no effect of the transgene) is correct.

The data obtained for DEL1f in the first experiment were confirmed in a second experiment with T2 plants. Four lines that had the correct expression pattern were selected for further analysis. Seed batches from the positive plants (both hetero- and homozygotes) in T1, were screened by monitoring marker expression. For each chosen event, the heterozygote seed batches were then retained for T2 evaluation. Within each seed batch an equal number of positive and negative plants were grown in the greenhouse for evaluation.

A total number of 120 DEL1f transformed plants were evaluated in the T2 generation, that is 30 plants per event of which 15 positives for the transgene, and 15 negatives.

Because two experiments with overlapping events had been carried out, a combined analysis was performed. This is useful to check consistency of the effects over the two experiments, and if this is the case, to accumulate evidence from both experiments in order to increase confidence in the conclusion. The method used was a mixed-model approach that takes into account the multilevel structure of the data (i.e. experiment—event—segregants). P-values are obtained by comparing likelihood ratio test to chi square distributions.

### Example 10

### Evaluation of DEL1f Transformants: Measurement of Yield-related Parameters

Upon analysis of the seeds as described above, the inventors found that plants transformed with the DEL1f gene construct had a higher seed yield, expressed as number of filled seeds, total weight of seeds and harvest index, compared to plants lacking the DEL1f transgene. It should be noted that the increase in yield was obtained in the absence of stress conditions. The results obtained for plants in the T1 generation are summarised in Table E:

TABLE E

| | % difference | p-value |
|---|---|---|
| Nr filled seeds | +38 | 0.0055 |
| Total weight seeds | +42 | 0.0039 |

These positive results were again obtained in the T2 generation. In Table F, data show the overall % increases for the number of filled seeds, total weight of seeds and harvest index, calculated from the data of the individual lines of the T2 generation, and the respective p-values. These T2 data were re-evaluated in a combined analysis with the results for the T1 generation, and the obtained p-values show that the observed effects were highly significant.

TABLE F

| | T2 generation | | Combined analysis |
|---|---|---|---|
| | % difference | p-value | p-value |
| Nr filled seeds | +14 | 0.0006 | 0.0001 |
| Total weight seeds | +12 | 0.0141 | 0.0002 |

### Example 11

### Downregulation of DEL1 Expression Leads to Enhanced Stress Resistance

The del1-1^{KO} allele of Arabidopsis thaliana (L.) Heynh. ecotype Columbia (Col-0) was obtained from the GABI-Kat T-DNA mutant collection (http://www.mpiz-koeln.mpg.de/GABI-Kat/GABI-Kat_homepage). For generation of DEL1^{OE} lines, the complete open DEL1 reading frame was cloned into the pK2GW7 vector [Karimi et al. (2002) Trends Plant Sci. 7, 193-195] with GATEWAY technology, resulting in the pKDEL1 vector, which was mobilized by the helper plasmid pRK2013 into the Agrobacterium tumefaciens C58C1 RifR strain harboring the pMP90 plasmid [Clough and Bent (1998) Plant J. 16, 735-743]. Plants were transformed by the floral dip method [S4]. Transgenic DEL1^{OE} plants were selected on kanamycin-containing medium.

Seeds from a DEL1 knock-out line (del1-1^{KO}), two independent DEL1 overexpressing lines (DEL1^{OE} #2 and DEL1^{OE} #4), and an untransformed wild-type (control) plant were sown in a mixture of soil and sand (4:1) and vernalized for 3 days at 4° C. for 3 days. Subsequently, the plants were subjected to a regime of 70 μmol.m⁻².s⁻¹ Photosynthetically Active Radiation, 14 h/10 h day/night cycle, and a temperature of 20° C./16-18° C. day/night in a relative humidity of 70-80%. After 18 days, the plants were exposed to UV-B light in a sunsimulator (GSF; National Research Center for Enviroment and Health, Munich, Germany), being a growth chamber mimicking the solar spectrum as it reaches the surface of the earth supplemented with an additional dose of UV-B (irradiance: 172 μmol.m⁻².s⁻¹ (±5%), 3.27 Wm⁻² (±5%). After 0, 3, 5, 7, and 9 hrs of UV-B exposure the plants were transferred back to the normal growth camber and their development was followed over time.

The different genotypes tested clearly responded differently to the UV treatment. UV-B treatment clearly affected the growth of the control plants in a dose-dependent manner. Leaves of plants treated for 5 hrs with UV-B showed necrosis and were reduced in size. This phenotype was even more outspoken for plants treated for 7 hrs with UV-B. Plants with increased DEL1 levels showed an even more severe response to UV-B, displaying already necrosis and inhibition of leaf growth at a dose of 3 hrs UV-B. By contrast, surprisingly, the del1-1^{KO} line displayed increased tolerance towards UV-B light. Whereas the size of control plants was clearly affected by a 3 hrs UV-B dose, del1-1^{KO} plants treated for 3 hrs were indistinguishable from the control plants. Also at a dose of 5 hrs UV-B the level of necrosis was severely reduced in comparison with the control plants.

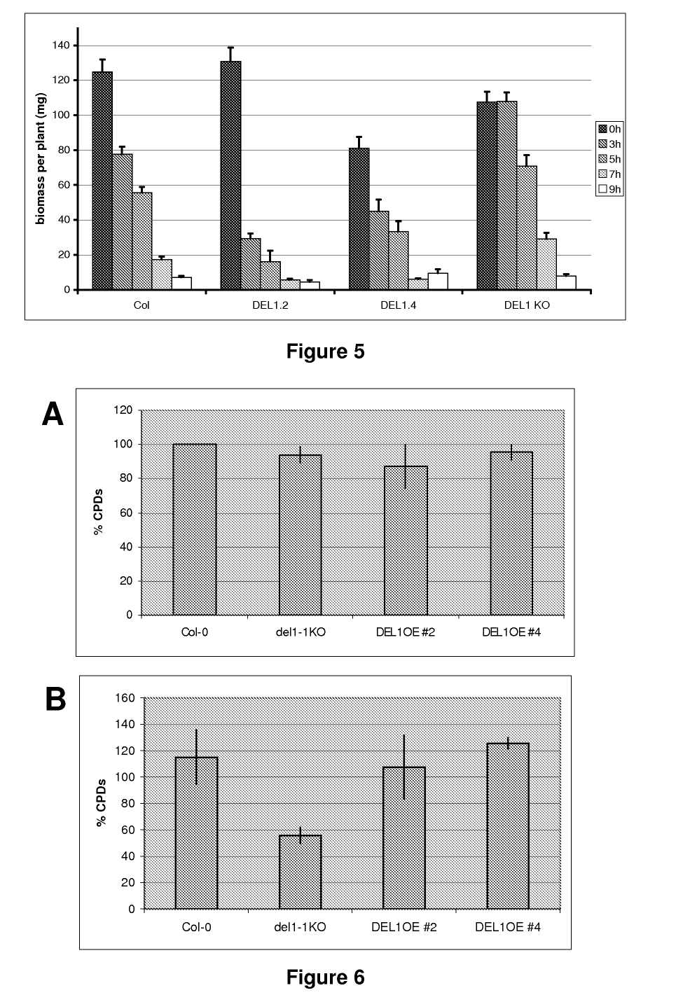

Visual observations were supported by biomass measurements (FIG. 5). The mass of 10-15 plants was measured, and averaged per plant. Whereas the untransfomed control plants showed a clear UV-B dose-dependent decrease in biomass per plant, del1-1^{KO} lines treated for less than 5 hrs displayed a biomass equal to that of the non-treated plants. Also at a dose of 5 hrs UV-B or longer, del1-1^{KO} plants performed better than control plants. In accordance with the observations that a decrease in DEL1 results in increased yield stability, the opposite phenotype was observed for the DEL1^{OE} #2 and DEL1^{OE} #4 lines, which were clearly more affected that control lines at any of the UV-B doses applied.

To identify the cellular basis underlying the increased yield stability of the DEL1^{KO} lines, epidermal cell numbers and sizes were determined by microscopy. The total blade area of the 5^{th} leaf was determined from digitized pictures taken directly with a charge-coupled device camera mounted on a binocular (Stemi SV11, Zeiss, Jena, Germany). Leaf blade area was determined from the images using the public domain image analysis program ImageJ (version 1.30; available on the website of the US National Institutes of Health, NIH). Cell density was determined from scanned drawing-tube images of the outline of at least 25 cells of the abaxial epidermis located at 25 and 75% from the distance between the tip and the base of the leaf after clearing the leaves in methanol followed by lactic acid treatment. From the images the average cell area and total number of cells per leaf was calculated by dividing the leaf area by the average cell area (averaged between the apical and basal positions). Comparative analysis of leaves treated for 5 hours with UV-B illustrated that total leaf blade area was decreased by 52% in control plants upon UV-B treatment. By contrast, del1-1^{KO} lines showed only a decrease of 15%. When counting cell numbers, an equal decrease in total cell number was observed for both genotypes (being −28% and −23% for control and del1-1^{KO} plants, respectively). By contrast, whereas the average cell size was declined with 33% in control plants (non-treated versus treated plants), cells of del1-1^{KO} plants were enlarged by 11% upon UV-B treatment. These data illustrate that the observed yield stability observed for the del1-1^{KO} plants upon stress treatment is specifically attributed to cell enlargement.

### Example 12

### del1-1^{KO} Plants Display Increased Levels of Photolyase PHR1 Expression and Activity

To uncover the molecular mechanism that confers UV-B resistance towards DEL1-deficient lines, a transcriptome analysis was performed. DEL1 knock-out (del1-1^{KO}), DEL1 overexpressing #4 (DEL1^{OE} #4), and untransformed wild-type (Col-0) seeds were sown on 1× Murashige and Skoog medium (Duchefa, Haarlem, The Netherlands) supplemented with 0.6% plant tissue culture agar (LabM, Bury, UK). After vernalization for 2 days at 4° C., plants were transferred to the growth chamber and grown at 22° C. and 65 μE·m²·sec⁻¹ irradiation in a 16 hr light/8 hr dark photoperiod, and a relative humidity of 50-60%. For the microarray analysis the most contrasting tissues were analyzed, being 8 day-old total seedlings for control (Col-0) vs. del1-1^{KO}, and the developing first leafpair for control vs. DEL1^{OE} #4, harvested at 8, 15, and 22 days after sowing. After statistic analysis, 16 genes were found to be display a complementary expression pattern in del1-1^{KO} vs. DEL1^{OE} #4 plants. One of the genes downregulated in the DEL1^{OE} #4 line and upregulated in del1-1^{KO} encoded for the type 2 CPD photolyase, representing an enzyme able to repair UV-caused DNA-damage in plant cells in the presence of white or blue light [Landry et al., 1997, PNAS 94, 328-332]. These data showed that plants with low or no DEL1 levels display an increased DNA repair activity after UV-B stress treatment. By contrast, DEL1 overexpressing plants, having a decreased level of type 2 CPD photolyase, will not be able to repair their DNA in the same extent as control or del1-1^{KO} plants. To test this hypothesis, seeds of the del1-1^{KO} line, two independent DEL1 overexpressing lines (DEL1^{OE} #2 and DEL1^{OE} #4), and an untransformed wild-type (Col-0) plant were sown in a mixture of soil and sand (4:1) and vernalized at 4° C. for 3 days. Subsequently, the plants were subjected to a regime of 70 μmol.m⁻².s⁻¹ PAR, 14 h/10 h day/night cycle, and a temperature of 20° C./16-18° C. day/night in a relative humidity of 70-80%. After 18 days, the plants were exposed to UV-B light (irradiance: 172 μmol.m⁻².s⁻¹±5%) in a sunsimulator (GSF; National Research Center for Environment and Health, Munich, Germany). After 5 hours of UV-B irradiation, the leaves were either harvested immediately, or left for recovery in white light (70 μmol.m⁻².s⁻¹ PAR) for 5 additional hours. The amount of photolyase activity was measured by determining the number of cyclobutylpyrimidine dimer (CPD) photoproducts by Elisa. Genomic DNA was extracted using a DNeasy Plant Mini Kit QIAGEN, Tokyo, Japan). A 50 μl aliquot of the extracted DNA at a concentration of 0.02 μg.ml⁻¹ was placed in each well. CPDs were detected with specific antibodies (TDM-2) as previously described (Tanaka et al, 2002, The Plant Journal 46, 317-326). When comparing the CPD level of the samples harvested directly after UV-B treatment, all genotypes displayed a similar DNA damage response (FIG. 6A). By contrast, when comparing samples allowed to recover in white light for 5 hours, nearly 45% of the DNA damage sites were repaired in the del1-1^{KO} mutant. Contrastingly, no DNA repair could be observed during the same time period in the control and the DEL1^{OE} overexpressing plants (FIG. 6B). These data show that del1-1^{KO} line performs better after UV-B treatment due to a faster DNA repair.

### Example 13

### Del1-1^{KO} Lines Display a Better Photosynthetic Performance After UV-B Irradiation

Seeds from a DEL1 knock-out line (del1-1^{KO}), two independent DEL1 overexpressing lines (DEL1^{OE} #2 and DEL1^{OE} #4), and an untransformed wild-type (Col-0) plant were sown in a standard commercial, peat-based compost, enriched with 1/3 John Innes 3 (loam based mix) and vernalized at 4° C. for one week. Subsequently, the plants were subjected to a regime of 100 μmol.m⁻².s⁻¹, 12 hrs photoperiod, and a temperature of 22° C. in a relative humidity of 60-80%. After 25 to 30 days, the plants were exposed to a short pulse of UV-B light. Efficiency of the photosystem II was measured before and 6 hours after the UV irradiation. When measured before irradiation, no significant difference could be found between the different genotypes. This indicated that all lines were healthy and unstressed before UV-treatment, and that the irradiation affected all the lines at the same extend. By contrast, when plants were submitted to a 48 hours-long UV-B treatment coupled with PAR, the del1-1^{KO} mutant had a slightly higher photosynthetic efficiency than the control, whereas the overexpressing lines had a photosynthetic performance reduced by 12% compared to Col-0. These data show that del1-1^{KO} lines outperformed control plants in their photosynthetic activity after UV-B treatment.

### Example 14

### DEL1-1^{KO} Plants also Display Enhanced Stress Resistance Under Constant UV-B Irradiation

To test whether the stress resistance of del1-1^{KO} holds true under a constant low level of UV-B (rather than in response to one acute dose), plants were grown for one week under a constant low flux of UV-B light. At the start of the experiment, no significant difference in rosette diameter was observed for the distinct genotypes. By contrasts, after one week of UV-B irradiation, the rosette diameter of the del1-1^{KO} plants (1.6 cm) was close to that of the untreated plants (1.8 cm), whereas those of the control (1.4 cm) and DEL1^{OE} (1.2 cm) were significantly more affected by the UV-B treatment.

1. A method for increasing plant yield and/or stress resistance relative to control plants, comprising decreasing expression in a plant of a nucleic acid encoding a DEL1 polypeptide and/or decreasing activity of a DEL1 polypeptide activity, and optionally selecting for plants having increased yield.

2. The method of claim 1, wherein said DEL1 protein comprises any one or more of the motifs of SEQ ID NO: 9, SEQ ID NO: 10, SEQ ID NO: 11, SEQ ID NO: 12 and SEQ ID NO: 13.

3. The method of claim 1, wherein said decreased expression and/or activity is effected by introducing a genetic modification in the locus of a gene encoding the DEL1 polypeptide.

4. The method of claim 3, wherein said genetic modification is effected by T-DNA activation, TILLING, site-directed mutagenesis, transposon mutagenesis, T-DNA insertion or directed evolution.

5. A method for increasing plant yield and/or stress resistance, relative to control plants, comprising introducing and expressing in a plant a DEL1 nucleic acid or a variant thereof.

6. The method of claim 5, wherein said nucleic acid or variant encodes an orthologue or paralogue of the DEL1 protein of SEQ ID NO: 8.

7. The method of claim 5, wherein said introduction results in RNA-mediated silencing of an endogenous DEL1 gene.

8. The method of claim 7, wherein said introduced DEL1 nucleic acid is of plant origin, preferably an endogenous DEL1 nucleic acid or substantially homologous to the endogenous DEL1 nucleic acid.

9. The method of claim 5, wherein said variant is a portion of the DEL1 nucleic acid or a sequence capable of hybridising to the DEL1 nucleic acid, which portion or hybridising sequence encodes a polypeptide comprising the sequence corresponding to the sequence C-terminally located to the DNA binding domains of a DEL1 protein and one or more of the signature sequences of SEQ ID NO: 9, SEQ ID NO: 10, SEQ ID NO: 11, SEQ ID NO: 12 and SEQ ID NO: 13.

10. The method of claim 9, wherein said portion encodes a DEL1 polypeptide that lacks part or all of the first and/or part or all of the second DNA binding region.

11. The method of claim 9, wherein said portion encodes a DEL1 protein that lacks a N-terminal part of the first DNA-binding domain.

12. The method of claim 9, wherein said portion encodes a polypeptide that is represented by SEQ ID NO: 2.

13. The method of claim 5, wherein said DEL1 nucleic acid or variant thereof is overexpressed in the plant.

14. The method of claim 5, wherein said DEL1 nucleic acid or variant thereof is operably linked to a seed-specific promoter.

15. The method of claim 14, wherein said seed-specific promoter is represented by SEQ ID NO: 5.

16. The method of claim 1, wherein said increased yield is increased seed yield.

17. The method of claim 16, wherein said increased seed yield comprises increased total weight of seeds and/or increased number of filled seeds.

18. The method of claim 1, wherein said increased stress resistance is increased resistance to abiotic stress or increased resistance to UV radiation.

19. A plant or a plant cell obtained by the method of claim 1, provided that said plant is not obtained by T-DNA insertion.

20. A construct comprising:
(i) a DEL1f nucleic acid or variant thereof,
(ii) one or more control sequences capable of driving expression of the nucleic acid sequence of (a); and
(iii) a transcription termination sequence.

21. The construct of claim 20, wherein said control sequence is a seed-specific promoter.

22. The construct of claim 21, wherein said seed-specific promoter is represented by SEQ TD NO: 5.

23. A construct for RNA-mediated silencing of an endogenous DEL1 gene comprising
(i) a DEL1 nucleic acid or variant thereof;
(ii) one or more control sequences capable of driving expression of the nucleic acid sequence of (a); and
(iii) a transcription termination sequence.

24. A method for increasing yield or increasing stress resistance of a plant, comprising transforming the construct according to claim 20.

25. A plant or a plant cell transformed with the construct of claim 20.

26. A method for the production of a transgenic plant having increased yield and/or increased stress resistance, comprising
(i) introducing and expressing in a plant or a plant cell a DEL1 nucleic acid or variant thereof; and
(ii) cultivating the plant cell under conditions promoting plant growth and development.

27. A transgenic plant having increased yield and/or increased stress resistance resulting from a DEL1 nucleic acid or a variant thereof introduced into said plant.

28. The transgenic plant or the plant cell of claim 27, wherein said plant is a monocotyledonous plant or a cereal.

29. Harvestable parts of the plant of claim 27.

30. Harvestable parts of a claim 29, wherein said harvestable parts are seeds.

31. Products directly derived from the plant of claim 25.

32. An isolated DEL1 polypeptide selected from the group consisting of:
(a) the polypeptide as given in SEQ ID NO 21,
(b) a polypeptide with an amino acid sequence which has at least 60% sequence identity to the amino acid sequence as given in SEQ ID NO 21, and
(c) a homologue, a derivative, and/or functional fragment of the protein as defined in (a).

33. An isolated nucleic acid sequence comprising:
(i) the nucleic acid sequence represented by SEQ ID NO: 20, or the complement strand thereof;
(ii) a nucleic acid sequence encoding the amino acid sequence of claim 27,
(iii) a nucleic acid sequence capable of hybridising (under stringent conditions) with the nucleic acid sequence of (i) or (ii) above, which hybridising sequence encodes a protein having yield increasing activity,
(iv) a nucleic acid which is an allelic variant to the nucleic acid sequences according to (i) to (iii),
(v) a nucleic acid which is a splice variant to the nucleic acid sequences according to (i) to (iii),
(vi) a nucleic acid sequence which has 60% sequence identity to SEQ ID NO: 20 or the complement thereof, and
(vii) a portion of the nucleic acid sequence according to any of (i) to (vi) above, which portion encodes a protein having yield increasing activity.

34-36. (canceled)

37. A breeding program for selecting a plant having increased yield and/or increased stress resistance, comprising using a DEL1 nucleic acid or variant thereof, or a DEL1 polypeptide or homologue thereof as a molecular marker and identifying the plant with increased yield and/or increased stress resistance.

38. The transgenic plant or the plant cell of claim 27, wherein said plant is sugar cane, rice, maize, wheat, barley, millet, rye, oats or sorghum.


SEQUENCE LISTING (31 sequences)

SEQ ID NO: 1 - DNA, 1044 bp, Arabidopsis thaliana
atggttgggcttgatgatgctgcctcgaaattaggagtggagagacgaagaatctatgatattgttaatgttctggagagtgttggggttttaacaagaagagcaaagaatcagtatacgtggaaagggttttccgcaattccaggagcattgaaggagctacaagaagagggggttaaggacacttttcatcgtttctatgtcaatgagaatgttaaaggatctgatgatgaggatgatgatgaagagtcttctcagcctcactctagtagccagactgatagttcaaaacctggttctcttccccaatcttcagatccctccaaaatagataaccgacgagagaaatctttaggattgcttactcagaactttatcaaactctttatttgctctgaagctattaggatcatctcccttgatgacgctgcaaaattactgcttggtgatgcccacaatacatcaataatgcgaactaaagtgaggcggctttatgatatagcaaatgtcttgtcgtcaatgaatctcatagagaagactcacaccttagattctaggaaaccagctttcaagtggttagggtacaatggtgagcctactttcacactgagcagcgatttgttgcaattggagtcaagaaaaagagctttcggaactgatattacaaacgtcaatgttaagagaagcaaatcatcatcttcgtcccaagaaaacgctacagagagaaggctaaagatgaaaaagcactcaacaccagagagttcttataacaaaagctttgatgttcatgaatcaagacatggatcaagaggaggttaccattttggaccttttgcaccaggcactggtacatatccaactgctggtttagaggataactctaggagagcttttgatgttgagaatctggattctgattaccgtccctcttaccaaaaccaagttttgaaagacctcttttcccattacatggatgcttggaagacatggttcagcgaagtcacccaggagaatccattacctaatacatcacaacaccgttag

SEQ ID NO: 2 - protein, 347 aa, Arabidopsis thaliana
MVGLDDAASKLGVERRRIYDIVNVLESVGVLTRRAKNQYTWKGFSAIPGALKELQEEGVKDTFHRFYVNENVKGSDDEDDDEESSQPHSSSQTDSSKPGSLPQSSDPSKIDNRREKSLGLLTQNFIKLFICSEAIRIISLDDAAKLLLGDAHNTSIMRTKVRRLYDIANVLSSMNLIEKTHTLDSRKPAFKWLGYNGEPTFTLSSDLLQLESRKRAFGTDITNVNVKRSKSSSSSQENATERRLKMKKHSTPESSYNKSFDVHESRHGSRGGYHFGPFAPGTGTYPTAGLEDNSRRAFDVENLDSDYRPSYQNQVLKDLFSHYMDAWKTWFSEVTQENPLPNTSQHR

SEQ ID NO: 3 - DNA, 54 bp, Artificial sequence
  primer prm00536
ggggacaagtttgtacaaaaaagcaggcttcacaatggttgggcttgatgatgc

SEQ ID NO: 4 - DNA, 52 bp, Artificial sequence
  primer prm00324
ggggaccactttgtacaagaaagctgggtctaacggtgttgtgatgtattag

SEQ ID NO: 5 - DNA, 1236 bp, Oryza sativa
ggtcagccaatacattgatccgttgccaatcatgcaaagtattttggctgtggccgagtgccggaattgataattgtgttctgactaaattaaatgaccagaagtcgctatcttccaatgtatccgaaacctggattaaacaatcctgttctgttctctagcccctcctgcatggccggattgtttttttgacatgttttcttgactgaggcctgtttgttctaaactttttcttcaaacttttaactttttcatcacatcagaacttttctacacatataaacttttaacttttccgtcacatcgttccaatttcaatcaaactttcaattttggcgtgaactaaacacaccctgagtcttttattgctcctccgtacgggttggctggttgagaataggtattttcagagagaaaatctagatattgggaggaacttggcatgaatggccactatatttagagcaattctacggtccttgaggaggtaccatgaggtaccaaaattttagtgtaaattttagtatctcattataactaggtattatgaggtaccaaatttacaatagaaaaaatagtacttcatggtactttcttaagtaccgtaaaattgctcctatatttaaggggatgtttatatctatccatatccataatttgattttgataagaaaaaatgtgagcacaccaagcatgtccatgaccttgcactcttggctcactcgtcaactgtgaagaacctcaaaaatgctcaatatagctacaggtgcctgaaaaaataactttaaagttttgaacatcgatttcactaaacaacaattattatctccctctgaaagatgatagtttagaactctagaatcattgtcggcggagaaagtaaattattttccccaaatttccagctatgaaaaaaccctcaccaaacaccatcaaacaagagttcaccaaaccgcccatgcggccatgctgtcacgcaacgcaccgcattgcctgatggccgctcgatgcatgcatgcttccccgtgcacatatccgacagacgcgccgtgtcagcgagctcctcgaccgacctgtgtagcccatgcaagcatccacccccgccacgtacaccccctcctcctccctacgtgtcaccgctctctccacctatatatgcccacctggcccctctcctcccatctccacttcacccgatcgcttcttcttcttcttcgttgcattcatcttgctagc

SEQ ID NO: 6 - DNA, 2332 bp, Artificial sequence
  expression cassette
ggtcagccaatacattgatccgttgccaatcatgcaaagtattttggctgtggccgagtgccggaattgataattgtgttctgactaaattaaatgaccagaagtcgctatcttccaatgtatccgaaacctggattaaacaatcctgttctgttctctagcccctcctgcatggccggattgtttttttgacatgttttcttgactgaggcctgtttgttctaaactttttcttcaaacttttaactttttcatcacatcagaacttttctacacatataaacttttaacttttccgtcacatcgttccaatttcaatcaaactttcaattttggcgtgaactaaacacaccctgagtcttttattgctcctccgtacgggttggctggttgagaataggtattttcagagagaaaatctagatattgggaggaacttggcatgaatggccactatatttagagcaattctacggtccttgaggaggtaccatgaggtaccaaaattttagtgtaaattttagtatctcattataactaggtattatgaggtaccaaatttacaatagaaaaaatagtacttcatggtactttcttaagtaccgtaaaattgctcctatatttaaggggatgtttatatctatccatatccataatttgattttgataagaaaaaatgtgagcacaccaagcatgtccatgaccttgcactcttggctcactcgtcaactgtgaagaacctcaaaaatgctcaatatagctacaggtgcctgaaaaaataactttaaagttttgaacatcgatttcactaaacaacaattattatctccctctgaaagatgatagtttagaactctagaatcattgtcggcggagaaagtaaattattttccccaaatttccagctatgaaaaaaccctcaccaaacaccatcaaacaagagttcaccaaaccgcccatgcggccatgctgtcacgcaacgcaccgcattgcctgatggccgctcgatgcatgcatgcttccccgtgcacatatccgacagacgcgccgtgtcagcgagctcctcgaccgacctgtgtagcccatgcaagcatccacccccgccacgtacaccccctcctcctccctacgtgtcaccgctctctccacctatatatgcccacctggcccctctcctcccatctccacttcacccgatcgcttcttcttcttcttcgttgcattcatcttgctagcatttaaatcaactagggatatcacaagtttgtacaaaaaagcaggcttcacaatggttgggcttgatgatgctgcctcgaaattaggagtggagagacgaagaatctatgatattgttaatgttctggagagtgttggggttttaacaagaagagcaaagaatcagtatacgtggaaagggttttccgcaattccaggagcattgaaggagctacaagaagagggggttaaggacacttttcatcgtttctatgtcaatgagaatgttaaaggatctgatgatgaggatgatgatgaagagtcttctcagcctcactctagtagccagactgatagttcaaaacctggttctcttccccaatcttcagatccctccaaaatagataaccgacgagagaaatctttaggattgcttactcagaactttatcaaactctttatttgctctgaagctattaggatcatctcccttgatgacgctgcaaaattactgcttggtgatgcccacaatacatcaataatgcgaactaaagtgaggcggctttatgatatagcaaatgtcttgtcgtcaatgaatctcatagagaagactcacaccttagattctaggaaaccagctttcaagtggttagggtacaatggtgagcctactttcacactgagcagcgatttgttgcaattggagtcaagaaaaagagctttcggaactgatattacaaacgtcaatgttaagagaagcaaatcatcatcttcgtcccaagaaaacgctacagagagaaggctaaagatgaaaaagcactcaacaccagagagttcttataacaaaagctttgatgttcatgaatcaagacatggatcaagaggaggttaccattttggaccttttgcaccaggcactggtacatatccaactgctggtttagaggataactctaggagagcttttgatgttgagaatctggattctgattaccgtccctcttaccaaaaccaagttttgaaagacctcttttcccattacatggatgcttggaagacatggttcagcgaagtcacccaggagaatccattacctaatacatcacaacaccgttag

SEQ ID NO: 7 - DNA, 1212 bp, Arabidopsis thaliana
atgtcagatctatcgccagaaagattcaaacttgccgttacttctccttcttccataccggaatcttcttcggctttacaattacaccattcctatagtcgcaaacagaaatctctcggacttctttgtaccaatttcttagctttgtataatcgagaagggattgaaatggttgggcttgatgatgctgcctcgaaattaggagtggagagacgaagaatctatgatattgttaatgttctggagagtgttggggttttaacaagaagagcaaagaatcagtatacgtggaaagggttttccgcaattccaggagcattgaaggagctacaagaagagggggttaaggacacttttcatcgtttctatgtcaatgagaatgttaaaggatctgatgatgaggatgatgatgaagagtcttctcagcctcactctagtagccagactgatagttcaaaacctggttctcttccccaatcttcagatccctccaaaatagataaccgacgagagaaatctttaggattgcttactcagaactttatcaaactctttatttgctctgaagctattaggatcatctcccttgatgacgctgcaaaattactgcttggtgatgcccacaatacatcaataatgcgaactaaagtgaggcggctttatgatatagcaaatgtcttgtcgtcaatgaatctcatagagaagactcacaccttagattctaggaaaccagctttcaagtggttagggtacaatggtgagcctactttcacactgagcagcgatttgttgcaattggagtcaagaaaaagagctttcggaactgatattacaaacgtcaatgttaagagaagcaaatcatcatcttcgtcccaagaaaacgctacagagagaaggctaaagatgaaaaagcactcaacaccagagagttcttataacaaaagctttgatgttcatgaatcaagacatggatcaagaggaggttaccattttggaccttttgcaccaggcactggtacatatccaactgctggtttagaggataactctaggagagcttttgatgttgagaatctggattctgattaccgtccctcttaccaaaaccaagttttgaaagacctcttttcccattacatggatgcttggaagacatggttcagcgaagtcacccaggagaatccattacctaatacatcacaacaccgttag

SEQ ID NO: 8 - protein, 403 aa, Arabidopsis thaliana
MSDLSPERFKLAVTSPSSIPESSSALQLHHSYSRKQKSLGLLCTNFLALYNREGIEMVGLDDAASKLGVERRRIYDIVNVLESVGVLTRRAKNQYTWKGFSAIPGALKELQEEGVKDTFHRFYVNENVKGSDDEDDDEESSQPHSSSQTDSSKPGSLPQSSDPSKIDNRREKSLGLLTQNFIKLFICSEAIRIISLDDAAKLLLGDAHNTSIMRTKVRRLYDIANVLSSMNLIEKTHTLDSRKPAFKWLGYNGEPTFTLSSDLLQLESRKRAFGTDITNVNVKRSKSSSSSQENATERRLKMKKHSTPESSYNKSFDVHESRHGSRGGYHFGPFAPGTGTYPTAGLEDNSRRAFDVENLDSDYRPSYQNQVLKDLFSHYMDAWKTWFSEVTQENPLPNTSQHR

SEQ ID NO: 9 - protein, 20 aa, Artificial sequence
  signature sequence 1
XXRKXKSLXXLCXXFXXXYX

SEQ ID NO: 10 - protein, 12 aa, Artificial sequence
  signature sequence 2
XLDXXAXXLGVE

SEQ ID NO: 11 - protein, 20 aa, Artificial sequence
  signature sequence 3
RXXKSLXXXXXXFXXLFXXX

SEQ ID NO: 12 - protein, 12 aa, Artificial sequence
  signature sequence 4
XXLXXAAXXXXX

SEQ ID NO: 13 - protein, 35 aa, Artificial sequence
  signature sequence 5
XKXRRLYDIANXLXSXXXIXKXXXXXXRXXXXXXX

SEQ ID NO: 14 - DNA, 1581 bp, Oryza sativa
cgcggacgcaccaagatccaatcctcgcgagcagaatcgatggcgacggcggcggtgatggctgctgtaccctcgtcttcgccggccgacgcggcggaggctgtggttatgacggaggcggtgccttctctcccgcaacgccagcagccggtgttcgtcgagggcagaggcgggaagctgcgtgaccacgcctacagccgcaagcagaagtcgctcggcctcctctgctccaatttcgtcgctctgtacaaccgcgacgacgtggagtctatcgggctggacgacgcggctaggaggctcggcgtggagaggcgccggatctacgacatcgtcaacgtgctcgagagcgtagggatcctcgtgaggaaggccaagaatcgttattcttggataggcttcggcggcgtcccaatggcattgcgagaactcaaggagagggcattgagagagaagtctggattggctcctctgcccgtggaggagccgtctgcagccattatgtcggatgacgaagatgaagataagatgggtgacgctgatggtgataccgagagcgagaagctgagtcaaccagttgacaacccttctgacaacaagccaggcgcacctcgctgccggcttagatctgaccataggaaggagaagtcgcttgggttgctcacgcagaatttcgtgaaactcttcctgaccatggaggttgacacaatctcacttgatgaagctgcaaagctgctacttggagaaggtcacgcagagaacagtatgagaactaaagtccggagactgtatgacattgctaacgtgctgtcatcactgaatttcattgataagatacaacaggcagactcaaggaaacctgcattccggtggttgggctcagcggggaaaccaaaagctgaaaatggtgtcacaatcgcagtacctccaccagggaagaccatatcgaacaagagagcatttgggactgaactcactaacattgacataaacagaagcagactggactcaacaatcccaaagaaagcaaagctgacactgagtggtggtgaaattttgaagaactgcaaattgtcagtgcagaaacagctcgggcagggtagcaagggtggttttgtttatgggcctttccaccctgctggtgcaagaaaacaagagcttgacaatggtaataaaggacacacagataatgttcaaaactgggagagccttgctgcttcatttcgaccacaataccagaaccaagcattgggcgatctttttgctcattatgtggaagcctggaaatcgtggtactctgaatttgcgcaaggcagcagcatgatgcagcagcactttggcatgcctgtcattaaccagtttttgtagtcaaatcattaatctcaagcactgtatttcttaccgcgtgcttgggattttacctgtacaatttgtcaagaaaatgagggaacaaagacatgacctaggcctaggatccattgtatttactcaatgcaggaaatatccaggctattcagtccaaaagataaactcgcctcattgttaggcatgtaactgttaattcatctagctataagctttcct

SEQ ID NO: 15 - protein, 441 aa, Oryza sativa
MATAAVMAAVPSSSPADAAEAVVMTEAVPSLPQRQQPVFVEGRGGKLRDHAYSRKQKSLGLLCSNFVALYNRDDVESIGLDDAARRLGVERRRIYDIVNVLESVGILVRKAKNRYSWIGFGGVPMALRELKERALREKSGLAPLPVEEPSAAIMSDDEDEDKMGDADGDTESEKLSQPVDNPSDNKPGAPRCRLRSDHRKEKSLGLLTQNFVKLFLTMEVDTISLDEAAKLLLGEGHAENSMRTKVRRLYDIANVLSSLNFIDKIQQADSRKPAFRWLGSAGKPKAENGVTIAVPPPGKTISNKRAFGTELTNIDINRSRLDSTIPKKAKLTLSGGEILKNCKLSVQKQLGQGSKGGFVYGPFHPAGARKQELDNGNKGHTDNVQNWESLAASFRPQYQNQALGDLFAHYVEAWKSWYSEFAQGSSMMQQHFGMPVINQFL

SEQ ID NO: 16 - DNA, 1461 bp, Zea mays
gcacgagccctcccagctcgcgcgccctcgatggatgcctccgccgccacccccgcccccgggccttccttctctggcgccgagtcctccgcggccgccgcccagccgccggcggaggctccgcagttgcgcgtccacggcgccggcagcggcagcggcgtcgcgcgagcctgccgccaccacgcgtacagccgcaagcagaagtcgctcggccttctctgctccaacttcgtggcgctgtacgaccgggaggacgtggaggtgattgggctggacgacgcggccaagcgtctcggcgtcgagcgacgccggatctacgacatagtcaacgttctcgagagcgtcgggattcttgtgcggagggccaagaatcggtatacatggctcggattcgggggagtccctgctgcgctgaaagaactcaaggagagggcgctaaggaagatgtccggatcaccggtgttactgtcaatggaggactcgtctactgccaacttatcagatgatgaggatgatgaaaaattgggcgatgctgatgaagatgctgagagcgagaagctcagccaacctgttgacaatacgtctgataagcctgacgcacccagctgccgccttagatctgatcatcggaaggagaagtcccttgggctcctcactcagaattttgtcaagctcttcctcaacatggaggttgggacaatctcacttgacgaagctgcaaggcttctccttggagagggacatgcagacagcaacatgagaacagccaaagttcgtcgattgtatgacattgccaatgtgctgtcttctttgaacctcattgagaagacgcagcaagcagacacaagaaaacctgcattccggtggctaggccaggcaaagcgaaagcaagataacaatgtcatggtttctgtacctccatccatgaaggcaatgcccaataagagatcatttggtactgatcttacaaacattgacaataagcgaggcaagttagactcagcagcggagaacaaagtcaagctcatgcagggtgctggtaacatagtgaagacttttgagaggcagctggtgcaagggaaaaggaatgactttgtttatgggcccttccaccctgctggtgcaaagaaacacgaaactgatgatcaaactgttaagcagcaggagaggaagaacattcaggactgggaaaaccttgctgtgtccttccgtccacaatatcagaatcaagcactgaatgatctttttggtcattatgtggaagcatggaaatcatggtacgtggatcttacccaggaaacggcatcatgaagcagaatgttggcaggtcagttgtaacttgtaagccgtttccagtagttagatcaactgagttgttgatgcttgcttagggaaccaccctgtacaagatggaataaaaacacgaaggcaattttgtctttcgactgtatttactcgatg

SEQ ID NO: 17 - protein, 426 aa, Zea mays
MDASAATPAPGPSFSGAESSAAAAQPPAEAPQLRVHGAGSGSGVARACRHHAYSRKQKSLGLLCSNFVALYDREDVEVIGLDDAAKRLGVERRRIYDIVNVLESVGILVRRAKNRYTWLGFGGVPAALKELKERALRKMSGSPVLLSMEDSSTANLSDDEDDEKLGDADEDAESEKLSQPVDNTSDKPDAPSCRLRSDHRKEKSLGLLTQNFVKLFLNMEVGTISLDEAARLLLGEGHADSNMRTAKVRRLYDIANVLSSLNLIEKTQQADTRKPAFRWLGQAKRKQDNNVMVSVPPSMKAMPNKRSFGTDLTNIDNKRGKLDSAAENKVKLMQGAGNIVKTFERQLVQGKRNDFVYGPFHPAGAKKHETDDQTVKQQERKNIQDWENLAVSFRPQYQNQALNDLFGHYVEAWKSWYVDLTQETAS

SEQ ID NO: 18 - DNA, 1512 bp, Ostreococcus tauri
atgaacggggatgggggggtggagagctcgcgcgcgtcgggcgggacggatgtcccgggcgaagacgcggcgcggacgcgcgcgacgccgagcgcgacgtctcggaaagataagtcgttgtggacgctgtgtgaacggtttttgacgatttatggagatggatcgaaggagagcgtgtcgctggacgatgcggcgacgcggctcggggtggagcgtcgaaggatttacgacgtcgcgaacgtgttggagagcgtggaggtgctcgagcgcaaggcaaagaatcagtacacgtggcacggcgtgcgaagactcccggagtgcctgaaacggctcaaggagagtggattgcgggaatttgggacggatgtcgagctcgatggaagcacgagcgagggacgtgacggggaaaaggaggatggaacggctcgaggcggcgacgcctccgatcgaagctctcccacaaactcgtcgacgatcaacttggacgcgaaacagcggggtgagaaggttactgggactaagttctttggacaggggcgattcgccgtctcgagcgccagttacgacagccggcgggagaagagtcttggcttgctgtcccaaaaattcgttcaactcttcctcgcgtcaaagatgaatgttgtcagtctggaaacggccgccaggattattatgggagaagacgacgacgatgaagccaagttgaagactaaaattcgtcgattgtacgatatcgccaacatcttgtgctccttgcgcctgattcgaaaggtacacgtgggcgagactagaaaaccagccttcctttggctacagcgagaaaactccatcgcggagctcatcgcgcagggcaaggggctgatgtggttcgataaattgaacgaagaggaagagatgcgtttacaggcgtccatcattgaatcaaaactcgacgggaacgaggtgttgatggttgacgcggagaataaacgacgaggcgcgttttacgattcgcagactaagtcgggatctaagcgtcctcgaggacgtccaagactgccgggtggtgacgtcgacgcgttgccttcatccgtacctccattaggcgcgtcttcgatgacgcccgaagaagcggcgaggttcaatcagttgttcgcgttcaccacgtcacaactcatggcacagtacccgctggacgtgcgcgcggatgtgaacagcatcatggcgcagagcgcgatgggtaacgcaaactaccttcatcttctcgcgcagtcttccgtggcccaagcgcacgcggcgcgacggatggagaacaccgcaaaaggaatgagctctgattccagcatcgacaacacggctatgttcgtgccgccacttcctgcgtttcccgcgatgcttccggcgtggggcatgtctggcttgtcttaccacagcaaccacatggaacacatgatgcggatgtacgaaacttctctggcaacgattagcgacaacgacccctcgcatccagatggaaagattgccgaacaataa

SEQ ID NO: 19 - protein, 503 aa, Ostreococcus tauri
MNGDGGVESSRASGGTDVPGEDAARTRATPSATSRKDKSLWTLCERFLTIYGDGSKESVSLDDAATRLGVERRRIYDVANVLESVEVLERKAKNQYTWHGVRRLPECLKRLKESGLREFGTDVELDGSTSEGRDGEKEDGTARGGDASDRSSPTNSSTINLDAKQRGEKVTGTKFFGQGRFAVSSASYDSRREKSLGLLSQKFVQLFLASKMNVVSLETAARIIMGEDDDDEAKLKTKIRRLYDIANILCSLRLIRKVHVGETRKPAFLWLQRENSIAELIAQGKGLMWFDKLNEEEEMRLQASIIESKLDGNEVLMVDAENKRRGAFYDSQTKSGSKRPRGRPRLPGGDVDALPSSVPPLGASSMTPEEAARFNQLFAFTTSQLMAQYPLDVRADVNSIMAQSAMGNANYLHLLAQSSVAQAHAARRMENTAKGMSSDSSIDNTAMFVPPLPAFPAMLPAWGMSGLSYHSNHMEHMMRMYETSLATISDNDPSHPDGKIAEQ

SEQ ID NO: 20 - DNA, 1161 bp, Medicago truncatula
atggctgcttcctcctccgatcctccttcacgacaccacacttacgaccgtaaacaaaaatcccttggcctcttatgcaccaagttcttgagcttgtataacaaagatgatgttcgtttaattggtctcgacgatgctgccgccaaattaggtgttgagagaagacggatctatgatattgtcaatgttctcgaaagcatcggggttcttgcaagaaaagccaagaatcagtatacctggaaaggttttgcggcaattcctgttgctctacaggagcttaaggaagagggtttatggcagaatctcaattcttcacaagaaggtgccaatgaagatgtgaaggtatcggatgaagaggatgaggatgaattgttatcccaaaccactggaagtcagggtgaatcattatcccaacccactggaagtcagaatgacaatctaaaccctaattccgcttttcccagatctttgaaaaatgacagaagggaaaaatctctggcgctgcttactcagaattttgtcaagctctttgtctgttccaacctggaaatgatatcgcttgatgatgcagcaaggttgttgcttggagatgcatataattcatcaacaatgagaacaaaagtcaggcgcctttatgatattgcaaacgtgctaacctccatgaaccttattgagaagacccataccacagatacaagaaaaccagcattcaggtggctaggcttaaaagggaagacattgaatgaggcatcactttacaattcaaaacaaaatgagtctaggaaaagggcgtttggaaatgatgtcacaaacataagctttgccaggaatagaatggacttgttcatgggcggggactttaagaagcaaaagacaatggaaaatgatagtggactatgtcaggaagatgtgaaacaaggcataaaacagacttcagcagctaactatcaatttggtccttttgctcctgcctttgtatccaaagctggaagctctgagaataaagtgaagcaggtgcatgactgggagagtctcgctactgaacattgccctcagtatcaaaaccaagctttgaaagaactttactctcattacatggaagcatggaaatcttggtactctgaagttgctgggaagaggtcaacgcaagttttgtagtaa

SEQ ID NO: 21 - protein, 385 aa, Medicago truncatula
MAASSSDPPSRHHTYDRKQKSLGLLCTKFLSLYNKDDVRLIGLDDAAAKLGVERRRIYDIVNVLESIGVLARKAKNQYTWKGFAAIPVALQELKEEGLWQNLNSSQEGANEDVKVSDEEDEDELLSQTTGSQGESLSQPTGSQNDNLNPNSAFPRSLKNDRREKSLALLTQNFVKLFVCSNLEMISLDDAARLLLGDAYNSSTMRTKVRRLYDIANVLTSMNLIEKTHTTDTRKPAFRWLGLKGKTLNEASLYNSKQNESRKRAFGNDVTNISFARNRMDLFMGGDFKKQKTMENDSGLCQEDVKQGIKQTSAANYQFGPFAPAFVSKAGSSENKVKQVHDWESLATEHCPQYQNQALKELYSHYMEAWKSWYSEVAGKRSTQVL

SEQ ID NO: 22 - DNA, 1510 bp, Triticum aestivum
ccctcctccaaggaccacaaaattcccccctccctaccctttccacctcgacatccaccggaaatggaccccgccgccgccgccggggctccggcggccgctgcgcagccaccgccccctcctcctcctccttacctgcccccgcggctggtcatcgacggcgccggaggcggctcgggcgccgccgtgagggcctgccggcaccacgcctacagccgcaagcagaagtccctcggcctcctctgctccaacttcgtggcgctgtacgaccgggacgacgtggagacggtggggctggacgacgccgccaggcggctcggcgtcgagaggcgccggatctacgacatcgtcaacgtgctcgagagcgtcgggattctcgtgaggagggccaagaatcggtacacatggatcggatttgagggcgtccctgccgcgctcaaggagcttaaggagaggacactgagagagatgtctggattagctccgccaccggaggaatcatctgctgccaatgtgtcggacgatgaagacgacgatgataaattgggcgatgcagatggggacgccgacagcgagaagcttagccagtccctcgacaatgcttctgataagcctaacgtgcccatgtgcccacctagatctgtagaccataggaaggagaagtcgcttgggctgctcacgcagaattttgtcaagctcttcctcaccatggaggttgagacagtctcacttgacgaggctgcaaggctgctccttggagagagacatgccgagagcaatatgagaactaaggttcgtcgactgtatgacatcgccaatgtgctatcttctttgaacctcattgagaagacacagcaggtggactccagaaaacctgcattccggtggctcggtcaggcaaagcgaaaggaaggtgccactgtcacggttgctttaccaccatccaggaagattatgtctagcaagagggcatttggtaccgacatcacaaacattgacaataagaggggcaagttagtcttggaaacagagaacaaacccaaactcatgcagggtggcagcagcatgttgaaaactttcgagagtcagctcgggcaagggaagagtagtggctttgtttatgggcccttccaccctgctggtgcaaggaaacatgaagttgatgatcagacagtgagggagaatgagatgaagaacattcaagactgggagagtctcgctgtttcattccgtccacagtaccaaaatcacgcgctgaatgatctttttggccattatgttgaagcatggaaatcatggtacttggatcttacacgggattcgacctcatgaaaggatggtttattttaggcaggtctgttgtaatcagttcttgtaggcagatcaactcatctgccgaggaccctagaaaggattcgacctgtacaagatgtcataaaagcacaaaggctcatttcgcctttgctcgatgtatttactcaatgcaaaaaagaaacggaataatgccaaa

SEQ ID NO: 23 - protein, 422 aa, Triticum aestivum
MDPAAAAGAPAAAAQPPPPPPPPYLPPRLVIDGAGGGSGAAVRACRHHAYSRKQKSLGLLCSNFVALYDRDDVETVGLDDAARRLGVERRRIYDIVNVLESVGILVRRAKNRYTWIGFEGVPAALKELKERTLREMSGLAPPPEESSAANVSDDEDDDDKLGDADGDADSEKLSQSLDNASDKPNVPMCPPRSVDHRKEKSLGLLTQNFVKLFLTMEVETVSLDEAARLLLGERHAESNMRTKVRRLYDIANVLSSLNLIEKTQQVDSRKPAFRWLGQAKRKEGATVTVALPPSRKIMSSKRAFGTDITNIDNKRGKLVLETENKPKLMQGGSSMLKTFESQLGQGKSSGFVYGPFHPAGARKHEVDDQTVRENEMKNIQDWESLAVSFRPQYQNHALNDLFGHYVEAWKSWYLDLTRDSTS

SEQ ID NO: 24 - DNA, 1530 bp, Oryza sativa
aaatctctcctcccgctcgccgaaaccctcgccgtcgccgcgccatggccgccgccgccgatgctcccccgcctcctccggaggtcgccccgcccgcgcccgcgcccgcccccgcgccggcgccatatcagccgccgcggctggcggtggccgacggagcgggaggtggcggcggcggcggcgggaagccgtgcaggcaccacgcgtacagccgcaagcagaagtcgctcggcctcctctgcaccaacttcgtggcgctgtacgaccgggaggacgtggagtcggtggggctggacgacgcggcgaggcggctgggcgtcgagaggcgccggatctacgacatcgtcaacgtgctcgagagcatcgggatgctcgtgaggagggccaagaatcggtatacgtggatcggcttcggtggagtccctgcggcgctcgcgaaactcaaggagatgtcactgagggcggtgtcaagcgtggcgtcaccgtcgctggatgaaacatctgctgctaatgtctcggatgatgaggatgatgacaagttagatgatgctgagggcgatgcggagagcgagaagctcagcctcagccagtccattgacaatccttctgataagcctgatgcacccccttgcaagcttcgatccgagcataggaaggagaagtcccttgggctgctcactcagaattttgtcaagctcttcctcaccatggagattgagacgatctcacttgatgaagccgcaaagcggctccttggagagggacatgcggcgaacaatatgagaaccaaagttcggcgattgtacgatattgccaatgtgctgtcttctttgaatcttattgagaagacacaacaggcggactcaagaaaacctgcattccggtggctgggccaggcaaaacggaatgaaggcgtcacggttgctttacccccaaccaagacgttgcctaacaagagagcatttggtactgatctgactaacattgacaataagaggggtaagttggactccacaatggagaacagaggcaagcccacgcaggatggtggcaacctattcaataatttgcagaggcaattagggcaagagaacaggagcgattttgcttatggccccttccaccctgctgttgcaaggaaacaagaacatggtaatcgcacagtacaagagaaggagaggaagagcattcaagactgggagaaccttgcttcttctttccgtccgcaatatcaaaatccaggactgaacgatctttttggccactacatggaagcaaggaggtcatggtactcggatctcaggcgagacagagcatcataaaaccagattcaggccggtccgttgtaaacaaaattcttgtaggcagataaactcatttgttgatgcatggttagggatttttgagttatacaagatgtcacaaaaacaaaggctagtgctggcctttgattgattgtatttactcagtgcaagaaattggaaagtcgtttcgtcaataatactaggatacctgattgtcgatttgttt

SEQ ID NO: 25 - protein, 425 aa, Oryza sativa
MAAAADAPPPPPEVAPPAPAPAPAPAPYQPPRLAVADGAGGGGGGGGKPCRHHAYSRKQKSLGLLCTNFVALYDREDVESVGLDDAARRLGVERRRIYDIVNVLESIGMLVRRAKNRYTWIGFGGVPAALAKLKEMSLRAVSSVASPSLDETSAANVSDDEDDDKLDDAEGDAESEKLSLSQSIDNPSDKPDAPPCKLRSEHRKEKSLGLLTQNFVKLFLTMEIETISLDEAAKRLLGEGHAANNMRTKVRRLYDIANVLSSLNLIEKTQQADSRKPAFRWLGQAKRNEGVTVALPPTKTLPNKRAFGTDLTNIDNKRGKLDSTMENRGKPTQDGGNLFNNLQRQLGQENRSDFAYGPFHPAVARKQEHGNRTVQEKERKSIQDWENLASSFRPQYQNPGLNDLFGHYMEARRSWYSDLRRDRAS

SEQ ID NO: 26 - DNA, 645 bp, Silene latifolia
gcacgagtcaccatcagaaaatcgatctttgcatcatggttatagtagaaagcagaaatctttgggtcttctttgttcaaatttcttgagattgtataatcgagacgatgtcgatttgattggtctagatgatgctgccagtaaattaggagtcgagcgaagaaggatctatgatattgttaatgtgttggagagtgtaggggtgttagcaagaaaagcgaaaaatcagtacacatggaagggctacaaggcaattcctaaggctcttgccttgttaaaggaagatggtttgaaggagaattttggtactgcagagggaagaagtagagtaaagtttcaggttttagactttgatgatgatgtagcttctaaccctgataccggaagccagtcggaggcaccaacacatagtggtgcttctaagcctgataacagaagggaaaagtcattggggcttctaacacaaaactttgtgaagctattcctttgttctgaggctgagctgatctctctggaggaagctgcaaagtgcttgcttggagatggtcttaatgcacaagtgatgcgaactaaagtgagacgattgtatgatattgccaatgtattatcctccatgcaactcattgaaaagactactcaaacaga

SEQ ID NO: 27 - protein, 214 aa, Silene latifolia
HESPSENRSLHHGYSRKQKSLGLLCSNFLRLYNRDDVDLIGLDDAASKLGVERRRIYDIVNVLESVGVLARKAKNQYTWKGYKAIPKALALLKEDGLKENFGTAEGRSRVKFQVLDFDDDVASNPDTGSQSEAPTHSGASKPDNRREKSLGLLTQNFVKLFLCSEAELISLEEAAKCLLGDGLNAQVMRTKVRRLYDIANVLSSMQLIEKTTQT

SEQ ID NO: 28 - DNA, 785 bp, Helianthus
cggggggacggatcgaactgttggattggatgacgccgcaacgaggctaggtgttgagagacggcggatttatgacattgttaatgttttggaaagcgttggcgtccttgtgaaaaaggcaaaaaacacgtatcattggttaggattgggggcaatacctaaggctttagagcagctaaaggaagaaggttttaggaataatgatgaacatgttgatgttaaatctggaaaggtttctgatgatgaagaagatgaaagagtttctagccatagtgtttctttacaggaaaaatcagatctagattcaatgcataaaacttcagggccgtttaaatcgggtagtgtaacggaaaatcggaaggaaaaatctttggggcttcttaccaagaatttcatcaagcttttcctatgcactaattcggatatgctttcgcttgatgacgctgcaaaaatattgcttggagatgctcagaatccgtcattgacccgaactaaagtcagacgcctatatgatattgctaatgtcttgtcttccatgcacttcatcgagaagatccatcacccagaaacccgaaagcccgcctttaggtggttgggaatggcaagtcacccaaatagccaaacgagatcagccactggtgtagctcatatagagtccaaaaaaagggcatttgggactgagcttacaaacatttgtttcaaaagaagcaagttgggtgaccaggaggtgaagctgtttgaccttcagagtcaacgcccgcctatcaaacccagcactcataatc

SEQ ID NO: 29 - protein, 261 aa, Helianthus
GGTDRTVGLDDAATRLGVERRRIYDIVNVLESVGVLVKKAKNTYHWLGLGAIPKALEQLKEEGFRNNDEHVDVKSGKVSDDEEDERVSSHSVSLQEKSDLDSMHKTSGPFKSGSVTENRKEKSLGLLTKNFIKLFLCTNSDMLSLDDAAKILLGDAQNPSLTRTKVRRLYDIANVLSSMHFIEKIHHPETRKPAFRWLGMASHPNSQTRSATGVAHIESKKRAFGTELTNICFKRSKLGDQEVKLFDLQSQRPPIKPSTHN

SEQ ID NO: 30 - DNA, 1125 bp, Glycine max
atgacttctttgtctacccatcacacctatagccgaaagcaaaaatctctcggcctcctctgcaccaattttctgagtttgtacaacaaagaaggtgtgcgcctggtcggtctcgatgacgcagcttcgcggttaggtgtagaaagacgtcggatctacgacatcgttaatgttttagagagtgtcggtgtgctaaccagaaaagctaagaatcagtatacttggaaaggattttgtgcaattcctgctgccttacaagaacttaaggatgaaggtttgaaggagaatcctggatcatttgatgactccaatgacaatgcaaaagtgtctgatgatgaggaggaggaagaagaaacatttcctaatattaacattgggagtcagagtgagaaggaaaatcctgattacactgctactgtcaaatcctccaagaatgaaaacagaagggaaaaatctctggcattgcttactcagaattttgtcaagctttttgtctgctctaattttgaaatgatctcccttgacgaagctgcaaagttgttgcttggaaatgccaacaatagaacaaaagtcagacgtctatatgatattgcgaatgtgttatcctccatgaacctaatcgagaagacccatacaacaaacacaagaaaaccagcattcaggtggttgggtgtgaggggaaaaacatggggtggatcagttgacttggctcaaaattcaaatgtcaaagagtcacggaaaaggatgtttggaactgacattggaaacataagttttaaaaggaacaaagttgatttgtccatggatggccagaattctaaaatgcaaaatcaacacgaaaatattagtccacgagctcagttagagaaaatcatcataaaaaaagatgctaagcagacttcagagagctatcagtttggcccttttgctccagcttatgtgcccaaagttggaacctctgaaaataatagtgtgaaaaaggtgcatgactgggacagcctcgcacaggagcatcgtcctcaatatcaaaaccaacctttaaaagatctattttcccactacatggaagcatggaaatcttggtactctgaagctgctgggaaaaagtcaacgaaaactttataa

SEQ ID NO: 31 - protein, 374 aa, Glycine max
MTSLSTHHTYSRKQKSLGLLCTNFLSLYNKEGVRLVGLDDAASRLGVERRRIYDIVNVLESVGVLTRKAKNQYTWKGFCAIPAALQELKDEGLKENPGSFDDSNDNAKVSDDEEEEEETFPNINIGSQSEKENPDYTATVKSSKNENRREKSLALLTQNFVKLFVCSNFEMISLDEAAKLLLGNANNRTKVRRLYDIANVLSSMNLIEKTHTTNTRKPAFRWLGVRGKTWGGSVDLAQNSNVKESRKRMFGTDIGNISFKRNKVDLSMDGQNSKMQNQHENISPRAQLEKIIIKKDAKQTSESYQFGPFAPAYVPKVGTSENNSVKKVHDWDSLAQEHRPQYQNQPLKDLFSHYMEAWKSWYSEAAGKKSTKTL